(19) United States
(12) Patent Application Publication
(10) Pub. No.: US 2025/0099402 A1
(43) Pub. Date: Mar. 27, 2025

(54) COMPOSITIONS OF ASPIRIN AND KETAMINE

(71) Applicant: Vitalis Analgesics LLC, Wilmington, DE (US)

(72) Inventors: Joseph Habboushe, New York, NY (US); Anish P. Dhanarajan, Catonsville, MD (US); Francesca K. Minale, Catonsville, MD (US)

(21) Appl. No.: 18/787,520

(22) Filed: Jul. 29, 2024

Related U.S. Application Data
(63) Continuation of application No. 18/219,446, filed on Jul. 7, 2023, now abandoned.
(60) Provisional application No. 63/359,006, filed on Jul. 7, 2022.

(51) Int. Cl. A61K 31/135; A61K 9/00; A61K 9/20; A61K 31/616; A61K 47/38; A61P 25/04

(52) U.S. Cl. CPC A61K 31/135; A61K 9/0053; A61K 9/2054; A61K 31/616; A61K 47/38; A61P 25/04

(57) ABSTRACT

Disclosed are pharmaceutical compositions having a portion of ketamine and/or aspirin for intraoral release and another portion of ketamine and/or aspirin for gastrointestinal release. The disclosed formulations and related administration approaches improve the bioavailability and efficacy of oral ketamine.

### CROSS REFERENCE TO RELATED APPLICATIONS

This application is continuation of U.S. application Ser. No. 18/219,446, filed on Jul. 7, 2023, which claims the benefit under 35 U.S.C. § 119(e) of U.S. Provisional Application Ser. No. 63/359,006, filed Jul. 7, 2022, the contents of which are incorporated by reference in its entirety into the present disclosure.

### BACKGROUND

Acute Pain is one of the most frequent chief complaints and the main reason for visiting the Emergency Department (ED). Recent literature indicates that 61-91% of patients are admitted to the ED due to a variety of acute painful syndromes. Musculoskeletal pain (MSK) affects one out of three adults and it is the most common source of serious long term pain and physical disability. Furthermore, studies have demonstrated that the frequency for analgesia for adults who received treatment for musculoskeletal pain in the ED is between 11-29%.

To complicate the issue of MSK pain management even further, the opioid epidemic spanning over 20 years in the USA and claiming over 400,000 deaths from unintentional prescription opioid overdose, has forced health care systems and hospitals across the nation to reduce the reliance on opioid analgesics and embrace the utility of non-opioid analgesia. Several classes of non-opioid analgesics such as acetaminophen, NSAID's (aspirin, ibuprofen, diclofenac) and ketamine have gained a great deal of attention as viable alternatives to opioids in management of acute MSK pain in the ED.

A combination of ketamine and aspirin for the treatment of acute MSK pain in the ED would confer pain relief while avoiding the use of opioids. There is a need to develop oral ketamine and aspirin formulations and oral ketamine and aspirin combination formulations.

### SUMMARY

It has been discovered that oral administration of ketamine achieved a remarkably higher bioavailability when the ketamine was partially released intraorally and delivered transmucosally and partially released through the gastrointestinal (GI) tract, as compared to intraoral or GI release alone. It has been discovered that co-administration of aspirin with ketamine can reduce certain side-effects associated with ketamine treatment, while also enhancing the therapeutic effect of ketamine.

In some embodiments, provided herein is a pharmaceutical composition comprising ketamine and aspirin, wherein, upon oral administration to a subject, a first portion disintegrates or dissolves intraorally providing rapid release of aspirin and ketamine, and a second portion is substantially more difficult than the first portion to disintegrate or dissolve intraorally but is ingestible and releases aspirin and ketamine in the gastrointestinal tract of the subject.

In some embodiments, provided is a pharmaceutical composition comprising ketamine, wherein, upon oral administration to a subject, a first portion disintegrates or dissolves intraorally providing rapid release of ketamine, and a second portion is substantially more difficult than the first portion to disintegrate or dissolve intraorally but is ingestible and releases ketamine in the gastrointestinal tract of the subject.

In some embodiments, provided is a pharmaceutical composition comprising aspirin, wherein, upon oral administration to a subject, a first portion disintegrates or dissolves intraorally providing rapid release of aspirin, and a second portion is substantially more difficult than the first portion to disintegrate or dissolve intraorally but is ingestible and releases aspirin in the gastrointestinal tract of the subject.

The pharmaceutical composition can be in the form of a tablet or capsule. In one embodiment, the second portion is enclosed within the first portion. In one embodiment, the first portion is a lozenge, buccal or sublingual composition.

In one embodiment, the second portion is compressed. In another embodiment, the hardness of the second portion is at least about 10 kilopascal (kp). In one embodiment, the second portion further comprises a pharmaceutically acceptable flavoring agent that is the same as the flavoring agent in the first portion. In another embodiment, the second portion further comprises a pharmaceutically acceptable flavoring agent that is different from the flavoring agent in the first portion. In some embodiments, the flavoring agent is optional and may not be present in the second portion.

### BRIEF DESCRIPTION OF THE DRAWINGS

FIG. 1 compares the pain reduction efficacies of different treatments.

FIG. 2 compares the sedation side effects of different treatments.

FIG. 3 compares the disassociation side effects of different treatments.

FIG. 4 compares the moderate/severe adverse events experienced in patients of different groups.

FIG. 5 compares the SERSDA2+ adverse events experienced in patients of different groups.

FIG. 6 compares the pain reduction in patients of different groups.

FIG. 7 compares the pain reduction in patients of different groups.

FIG. 8 compares the SERSDA2+ adverse events experienced in patients of different groups.

FIG. 9 compares the pain reduction in patients of different groups.

FIG. 10 compares time to reach 50% pain reduction in patients of different groups.

FIG. 11 presents the pain reduction results of the different treatments tested in Example 7.

FIG. 12, panel A shows a representation of a pharmaceutical composition which is a tablet in tablet compressed dosage form. Panel B shows a representation of a pharmaceutical composition comprising a first composition and a second composition adjacent to, or in proximity to, each other.

FIG. 13 shows an embodiment where beads or granules are present within the pharmaceutical composition/dosage form.

### DETAILED DESCRIPTION

### A. Definitions

Unless defined otherwise, the terms used herein are intended to have their ordinary meaning in the art.

All numerical designations, e.g., pH, temperature, time, concentration, and weight, including ranges, are approximations that typically may be varied (+) or (−) by increments of 0.1, 1.0, 10.0, or 100.0 as appropriate. It is to be understood, although not always explicitly stated, that all numerical designations are preceded by the term “about”.

“About” will be understood by persons of ordinary skill in the art and will vary to some extent on the context in which the term is used. If there are uses of the term which are not clear to persons of ordinary skill in the art given the context in which it is used, “about” will mean up to plus or minus 10%, or 5%, or 2% or 1% or 0.5% of the particular term.

As used herein, the term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As used in the specification and claims, the singular form “a”, “an”, and “the” includes plural references unless the context clearly dictates otherwise.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

As used herein, “compressed” dosage form (e.g., “compressed portion”), refers to a dosage form comprising a compressed powder. For example, a compressed portion may be formed using a rotary tablet press or other similar machinery known to one of skill in the art.

As used here, “disintegrates or dissolves intraorally” refers to that a majority of a composition or a portion of a composition, such as a tablet or a capsule, breaks apart into smaller particles intraorally. The majority, in one embodiment, means at least about 50%, or alternatively at about 60%, or 70%, or 80%, or 90%, or 95%, or 98%, or 99%.

As used herein, “bilayer” compressed dosage form (e.g., “bilayer tablet”) refers to a single compressed dosage form comprising two layers. A bilayer compressed dosage form can be made in a single compression step. Likewise, a “trilayer” compressed dosage form (e.g., “trilayer tablet”) refers to a single compressed dosage form comprising three layers.

As used herein, “wet granulation” refers to a process known in the pharmaceutical arts that involves forming granules by the addition of a liquid, such as purified water, alcohol, or a binder solution.

“Controlled release formulation” refers to a formulation in which the active agent is included within a matrix, which matrix can be either insoluble, soluble, or partly soluble. Controlled release matrix formulations of the insoluble type are also referred to as insoluble polymer matrices, swellable matrices, or lipid matrices depending on the components that make up the matrix. Controlled release matrix formulations of the soluble type are also referred to as hydrophilic colloid matrices, erodible matrices, or reservoir systems. Controlled release formulations of the present disclosure refer to formulations comprising an insoluble matrix, a soluble matrix or a combination of insoluble and soluble matrices in which the rate of release is slower than that of an uncoated non-matrix or immediate release formulations or uncoated normal release matrix formulations. Controlled release formulations can be coated with a control releasing coat to further slow the release of aspirin from the controlled release matrix formulation. Such coated controlled release matrix formulations can exhibit modified-release, controlled-release, sustained-release, extended-release, prolonged-release, delayed-release, or combinations thereof, of aspirin. Examples of controlled release forms of aspirin include Slo-Aspirin® available from Upsher Smith Laboratories, Inc. (Maple Grove, MN).

“Controlled release coat” refers to a functional coat which can, for example, include at least one pH independent or pH dependent (such as for example enteric or reverse enteric types) polymer, soluble or insoluble polymer, lipids or lipidic materials, or combinations thereof, which, when applied onto a formulation can slow (for example, when applied to an immediate release formulation or a normal release matrix formulation), further slow (for example when applied to a controlled release matrix formulation), or modify the rate of release of aspirin.

“Core composition” refers to a composition that is then further coated with one or more coatings (e.g., 1 to 5 coatings). In some embodiments, a core composition is a core tablet, i.e. a compressed tablet solid dosage form comprising active ingredients and excipients. In some embodiments, a core composition is a capsule filled with active ingredients and excipients.

“Excipient” refers to a pharmacologically inactive substance used with the active agents or drugs of a medication or a formulation. Excipients are also sometimes used to bulk up formulations that contain very potent active ingredients, to allow for convenient and accurate dosage. In addition to their use in the unit dose forms, excipients can be used in the manufacturing process to aid in the handling of the active substance concerned. Depending on the route of administration, and form of medication, different excipients may be used. Examples of an excipient includes, without limitation, one or more of the following: an additive, an anti-foaming agent, a binder, a chemical stabilizer, a coloring agent, a diluent, a disintegrating agent, an emulsifying agent, a filler, a flavoring agents, a glidant, a lubricant, a pH modifier, a plasticizer, a solubilizer, a swelling enhancer, a spheronization aid, a solubility enhancer, or a suspending agent.

“Immediate release formulation” refers to a formulation from which the drug is released without any substantial delay and substantially at once.

“Patient” or “subject” refers to mammals, including humans and animals, such as simians, cattle, horses, dogs, cats, and rodents having the need to take aspirin.

“Pharmaceutically acceptable salt” refers to pharmaceutically acceptable salts derived from a variety of organic and inorganic counter ions well known in the art that include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use, 2002, incorporated herein by reference.

“Plasticizer” refers to a compound capable of plasticizing or softening a polymer or a binder. Plasticizers can broaden the average molecular weight of a polymer in which they are included thereby lowering its glass transition temperature or softening point. Plasticizers also can reduce the viscosity of a polymer. The use of plasticizers is optional, but they can be included in a formulation to modify the properties and characteristics of the polymers used in the coat(s) or core of the formulation for convenient processing during manufacture of the coat(s) and/or the core of the formulation. Once the coat(s) and/or core has been manufactured, certain plasticizers can function to increase the hydrophilicity of the coat(s) and/or the core of the formulation in the environment of use. During manufacture of the coat(s) and/or core, the plasticizer can lower the melting temperature or glass transition temperature (softening point temperature) of the polymer or binder.

“Solid formulation” refers to a formulation that is neither liquid nor gaseous. Solid formulations include tablets, powders, microparticles, capsules, matrix forms, suppositories, sachets, troches, patches, buccal formulations, sublingual formulations and lozenges. Solid formulations in the form of capsules contain a solid composition within a capsule that can be made of gelatin or other encapsulating material. Liquid formulations include liquid suspensions and elixirs and may be present in a capsule.

“Substrate” in the context of spray coating refers to a core composition (e.g., a core tablet, a core capsule) having one or more coats described herein deposited thereon.

“Substantially free” of e.g., a solvent, e.g., water, alcohol, means that any residual solvent is less than 5%, less than 4%, less than 3%, less than 2%, less than 1%, or less than 0.5%. By way of example only, where an aspirin coat is substantially free of water, the aspirin coat comprises less than 5%, less than 4%, less than 3%, less than 2%, less than 1%, or less than 0.5% of any residual water after the sprayed coat is dried. By way of example only, where a ketamine coat is substantially free of alcohol, the ketamine coat comprises less than 5%, less than 4%, less than 3%, less than 2%, less than 1%, or less than 0.5% of any residual alcohol after the sprayed coat is dried.

“Swelling enhancer” refers to an excipient that swells rapidly resulting in an increase in the size of the tablet. At lower concentrations, these excipients can be used as super disintegrants; however at higher concentrations, e.g., at concentrations above about 5% w/w, these excipients function as swelling enhancers and increase the size of the matrix formulation.

“Therapeutically effective amount” refers to an amount of the drug that, when administered to a patient, will have the intended therapeutic effect, e.g., alleviation, amelioration, palliation or elimination of one or more manifestations of cancer or other hyperproliferative disease in the patient. A therapeutic effect does not necessarily occur by administration of one dose, and may occur only after administration of a series of doses. Typically, cancer drugs are administered in a repeating series of doses, and in certain instances each series may be referred to as a “cycle” of therapy. Thus, a therapeutically effective amount may be administered in one or more administrations.

“Treating” or “treatment of” a condition or patient refers to taking steps to obtain beneficial or desired results, including clinical results. For purposes of this disclosure, beneficial or desired clinical results include, but are not limited to, in intended treatment purpose of ketamine such as pain, anesthesia, asthma, depression, or other beneficial results including reduction of side effects.

### B. Oral Formulations

Ketamine is commonly used for anesthesia. It induces a trance-like state while providing pain relief, sedation, and memory loss. Other uses include sedation in intensive care and treatment of pain and depression. Heart function, breathing, and airway reflexes generally remain functional. Common side effects include agitation, confusion, or hallucinations as the medication wears off. Elevated blood pressure and muscle tremors are also common. Spasms of the larynx may occur, but relatively infrequently.

Ketamine can be absorbed by intravenous, intramuscular, oral, and topical routes due to both its water and lipid solubilities. In medical settings, ketamine is usually injected intravenously or intramuscularly. Oral ketamine, however, is easily broken down by bile acids, and hence has a low bioavailability. Bioavailability through the oral route reaches 17 to 29%. By contrast, bioavailability through intramuscular injection is about 93%. The onset of action of ketamine is seconds intravenously and 1 to 5 minutes intramuscularly, but 15 to 30 minutes orally. Moreover, maximal concentrations of ketamine are reached in 1 to 3 minutes intravenously, and 5 to 15 minutes intramuscularly, but 30 minutes orally.

Aspirin is a salicylate used to treat minor aches and pains due to various conditions such as headache, toothache, menstrual cramps, or muscle aches. Aspirin relieves pain and decreases swelling. Aspirin is rapidly absorbed from the gastrointestinal (GI) tract when administered as a solution, and somewhat more slowly when administered in tablets.

There is a need to develop oral formulations of ketamine with improved bioavailability and faster action. There is also a need to reduce the side-effects of ketamine when the medication wears off.

It has been discovered that oral administration of ketamine achieved a remarkably higher bioavailability when the ketamine was partially released intraorally and delivered transmucosally and partially released through the gastrointestinal (GI) tract, as compared to intraoral or GI release alone. Further, certain desired effects of ketamine, such as pain (acute pain, acute on chronic pain, chronic pain) reduction, suppression of depression, reduction of fatigue (e.g., multiple sclerosis fatigue), reduction of suicidality, treatment of asthma/reactive airway, treatment of cannabinoid hyperemesis syndrome exacerbations, and treatment of Alzheimer's/dementia can be further enhanced by co-administration with aspirin. At the same time, co-administration with aspirin can reduce the undesired effects of ketamine, including its sedation effect and addiction potential.

Described herein are dual release oral compositions comprising aspirin and/or ketamine which provide an initial intraoral release of ketamine and/or aspirin, followed by gastrointestinal release of ketamine and/or aspirin. Co-formulation of drugs presents challenges because the drugs may react with each other, or the drugs may be incompatible with certain excipients or with processes used in the manufacture of pharmaceutical formulations.

In the course of preparing combination dosage forms of aspirin and ketamine described herein, an unexpected problem arose because ketamine and aspirin could not be spray coated out of the same solution. It was found that ketamine unexpectedly reacted with alcohol solvents leading to impurities. Therefore, aqueous solutions were needed for spray coating ketamine. Aspirin however hydrolyses in aqueous solutions and therefore aspirin was could not be spray coated from alcoholic solutions.

Accordingly the combination dosage forms described herein comprise multilayer coatings for the intraoral release component. In achieving the intraoral release of ketamine and aspirin, contact of ketamine with any residual alcohol from an aspirin spray dried coating, and contact of aspirin with any residual water from a ketamine spray dried coating is minimized by using a seal coat between a ketamine coating and an aspirin coating. Optionally multiple coatings of ketamine and/or aspirin may be layered onto a core composition comprising ketamine and aspirin. The coatings are formulated for burst release in the oral cavity and/or transmucoal absorption. The coatings dissolve or disintegrate in the oral cavity while the core composition passes into the GI tract, and in this manner a dual-mode release of ketamine and aspirin is achieved where a first portion is released intraorally and a second component is released in the GI tract. Optionally a third mode of release can be achieved by using suitable enteric coatings.

### C. Administration of Oral Formulations

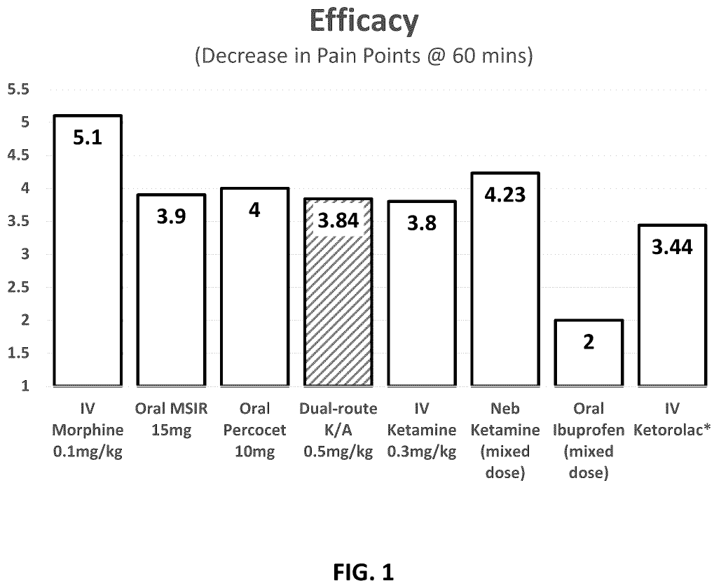

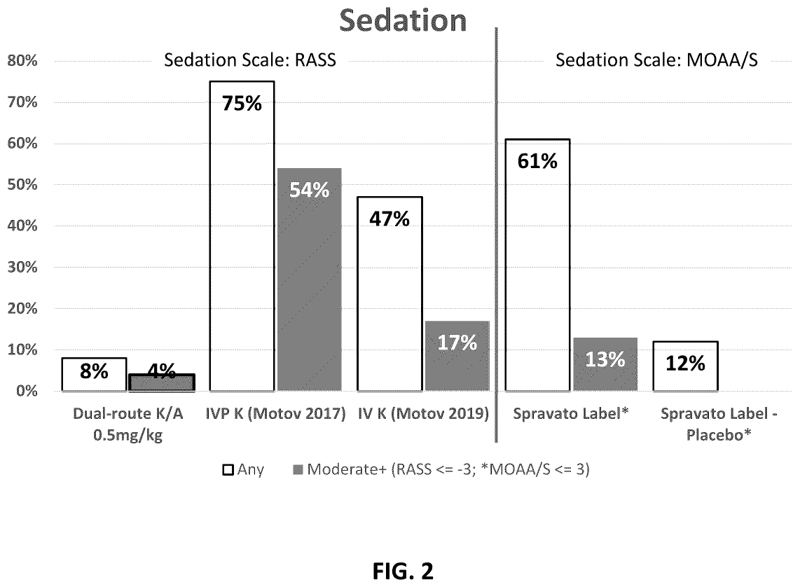

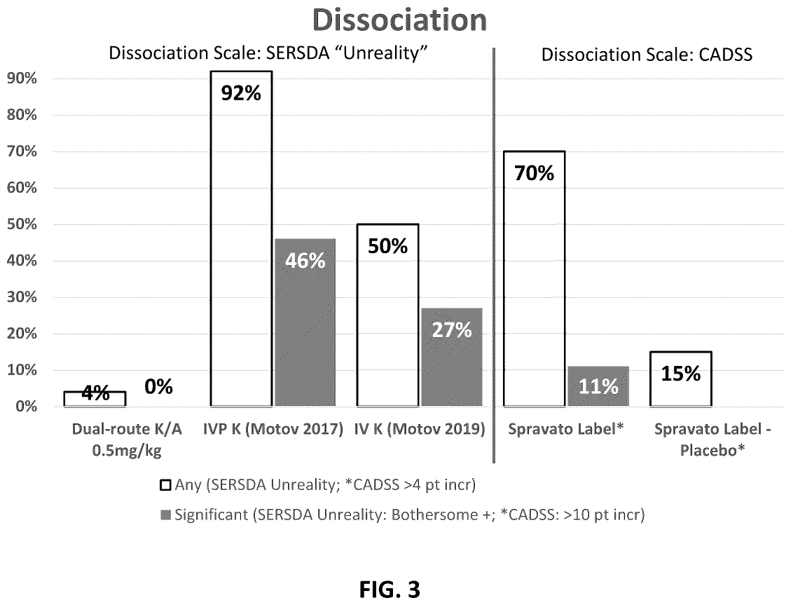

As demonstrated in the accompanying experimental examples, and in co, when ketamine was administered with aspirin through two different route of administration (intraoral and oral) its efficacy was even better than intravenously administered ketamine and was close to orally administered MSIR (Morphine) (FIG. 1), and furthermore significantly better than intravenously administered ketamine if corrected for expected bioavailability of oral ketamine (oral ketamine is about 18-20% bioavailable, while IV ketamine is about 100% bioavailable). Meanwhile, the sedation and dissociation side effects were considerably reduced as compared to intravenously administered ketamine (FIG. 2-3).

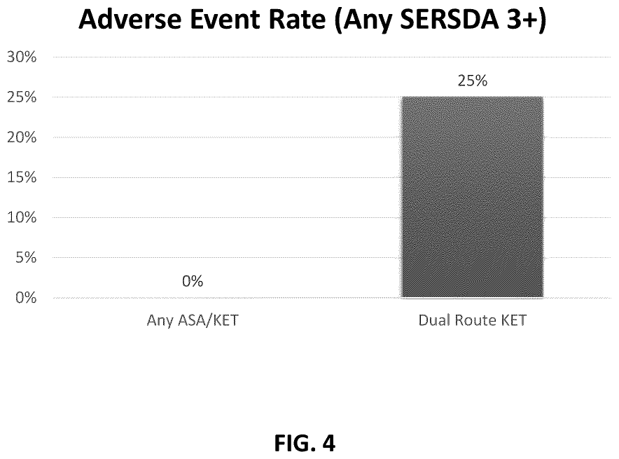

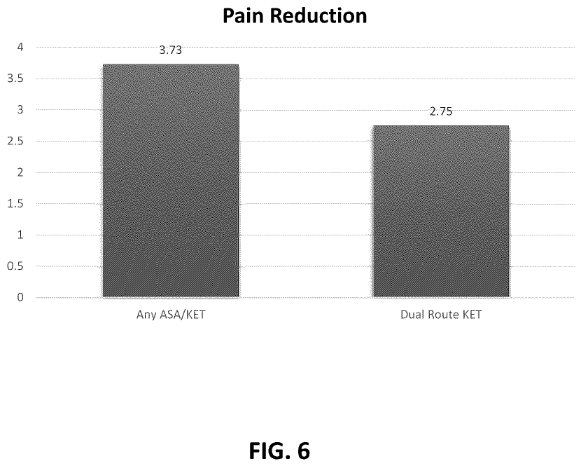

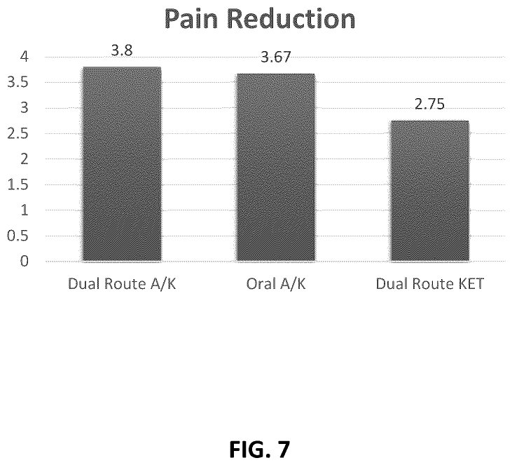

It is commonly known that oral ketamine has limited efficacy and, more importantly, ketamine from any route when administered at efficacious doses have side effects limiting outpatient use, and thus a narrow therapeutic window outside of the monitored setting. Accordingly, oral ketamine is not approved for outpatient use. It was further investigated whether a simple combination of oral aspirin and oral ketamine (oral aspirin/ketamine), or a dual route of ketamine (dual route ketamine) would have acceptable safety margin for outpatient use. Unexpectedly, the simple oral aspirin and ketamine administration resulted in a 3.67-point pain reduction (FIG. 7), close to the dual route aspirin/ketamine administration. The dual route ketamine, likewise, reached a 2.75-point reduction (FIG. 6) and only 25% SERSDA3+ adverse even rate (FIG. 4). Such a safety margin is significantly improved as compared to oral ketamine alone.

It is contemplated that such greatly improved efficacy and reduced side effects were at least in part attributed to the dual modes of administration of ketamine, one of which includes intraoral administration for release in the mouth and absorption through the mucosal membrane. The other portion of the ketamine was released in the GI tract. The partial intraoral release and partial GI release of ketamine are contemplated to achieve a synergistic effect in increasing the bioavailability and efficacy of ketamine. GI-absorbed ketamine has a different metabolic profile from intraorally absorbed ketamine which directly enters into the blood stream. This is likely due to the first-pass metabolism of the liver for medication absorbed through the GI tract.

When administered orally, ketamine undergoes first-pass metabolism, where it is biotransformed in the liver by CYP3A4 (major), CYP2B6 (minor), and CYP2C9 (minor) isoenzymes into norketamine (through N-demethylation) and ultimately dehydronorketamine. Intermediate in the biotransformation of norketamine into dehydronorketamine is the hydroxylation of norketamine into hydroxynorketamine by CYP2B6 and CYP2A6. As the major metabolite of ketamine, norketamine is one-third to one-fifth as potent as an anesthetic, and plasma levels of this metabolite are three times higher than ketamine following oral administration.

Ketamine given directly into the blood stream results in a fast peak of serum ketamine concentration, which immediately begins to drop. The immediate drop is due to the fast metabolism of ketamine into its primary metabolite, norketamine, resulting in high serum levels of norketamine.

Accordingly, the current data suggest that ketamine absorbed directly into the blood stream (such as oral mucosally absorbed) hits a peak serum concentration relatively quickly, within minutes. Ketamine given concurrently through the GI, on the other hand, has a serum peak concentration about 15-20 minutes later. Therefore, when just focusing on the ketamine serum levels, a combination of direct-absorption and GI-absorption will “smooth out” the ketamine serum concentration over time: the direct absorption getting serum levels high early, and the GI absorption keeping serums level high later. This higher and wider serum level of ketamine result in a more pronounced effect.

It is further contemplated that the greatly improved efficacy and reduced side effects of ketamine were also attributed to the combinatory use of ketamine with aspirin. A combination of ketamine and aspirin is contemplated to confer multimodal analgesia, with the contributions of aspirin and ketamine to an opioid sparing effect. It is further contemplated that such effect of aspirin would be observed with other modulators of glutamate ionotropic receptor NMDA type subunit 2A (GRIN2A), such as nicotine, propofol, melatonin, and gangliosides. Gangliosides are sialic acid-containing glycosphingolipids, among which tetrasialoganglioside GQ1b is an example (alpha-N-acetylneuraminosyl-(2->8)-alpha-N-acetylneuraminosyl-(2->3)-beta-D-galactosyl-(1->3)-N-acetyl-beta-D-galactosaminyl-(1->4)-[alpha-N-acetylneuraminosyl-(2->8)-alpha-N-acetylneuraminosyl-(2->3)]-beta-D-galactosyl-(1->4)-beta-D-glucosyl-(1 <->1′)-N-(octadecanoyl)-sphing-4-enine; PubChem ID: 10887808).

Oral administration of ketamine results in decreased ketamine and increased nor-ketamine concentrations in serum. Consequently, oral ketamine's first-pass effect from hepatic metabolism of ketamine to nor-ketamine may help maintain analgesic potency while simultaneously decreasing side effects when compared to the IV form.

Aspirin is a prototype of non-steroidal anti-inflammatory drugs (NSAIDs), and member of the family of salicylates that have in common salicylic acid as the active agent. The pharmacological properties of aspirin are similar to those of salicylates, but also to the biological actions attributed to salicylate itself, and it has other independent effects due to its reactive acetate group. Both components, salicylate and acetate groups, are biologically active and act independently of each other at different sites. Aspirin is a safe and well-understood non-steroidal anti-inflammatory drug (NSAID). It has certain and clinically accepted analgesic properties. It is a non-selective and irreversible NSAID that inhibits an activity of both cyclooxygenase-1 and 2 and blocks the synthesis of prostaglandins and thromboxanes.

An oral combination drug of aspirin (or another GRIN2A activator such as nicotine, propofol, melatonin and GQ1b) and ketamine (e.g., combination of two administration route) would facilitate the shift from IV opioids to a non-IV therapy for patients with acute MSK pain. This combination has a potential to provide effective analgesia with reduced side effects.

It is contemplated that the synergistic effects between intraoral ketamine and oral ketamine, and between ketamine and aspirin, is applicable to analogs and metabolites of ketamine and other N-methyl-D-aspartate (NMDA) receptor antagonists, and applicable to other NSAIDs.

Example NMDA receptor antagonists include, without limitation, ketamine, dextromethorphan, memantine, and amantadine, as well as opioids methadone, dextropropoxyphene, and ketobemidone. Non-limiting examples of ketamine metabolites include norketamine, hydroxynorketamine (HNK), dehydronorketamine (DHNK), and 6-hydroxyketamine (HK).

Accordingly, one embodiment of the present disclosure provides oral formulations of an NMDA receptor antagonist (e.g., ketamine or a metabolite or analog thereof) suitable/acceptable for outpatient use. In some embodiments, provided is a pharmaceutical composition comprising a first portion comprising a first amount of an NMDA receptor antagonist (e.g., ketamine or a metabolite or analog thereof), and a second portion comprising a second amount of an NMDA receptor antagonist (e.g., ketamine or a metabolite or analog thereof), wherein, upon oral administration to a subject, the first portion disintegrates or dissolves intraorally providing rapid release of the NMDA receptor antagonist of the first portion, and the second portion is substantially more difficult than the first portion to disintegrate or dissolve intraorally but is ingestible and releasable in the gastrointestinal tract of the subject.

In one embodiment, the NMDA receptor antagonist (e.g., ketamine or a metabolite or analog thereof) of the first portion is at an amount lower than the regular dose of the NMDA receptor antagonist (e.g., 100-400 mg), such as but not limited to, from about 10 mg to about 300 mg. In one embodiment, the amount of the NMDA receptor antagonist of the first portion is at least about 10 mg, or least about 20 mg, 30 mg, 40 mg, 50 mg, or 100 mg. In another embodiment, the amount of the NMDA receptor antagonist of the first portion is no more than about 50 mg, 75 mg, 100 mg, 125 mg, 150 mg, 175 mg, 200 mg, 225 mg, 250 mg, 275 mg or 300 mg. In one embodiment, the NMDA receptor antagonist in the second portion is at an amount lower than the regular dose of the NMDA receptor antagonist (e.g., 100-400 mg), such as but not limited to, from about 10 mg to about 300 mg. In one embodiment, the amount of the NMDA receptor antagonist of the first portion is at least about 10 mg, or least about 20 mg, 30 mg, 40 mg, 50 mg, or 100 mg. In another embodiment, the amount of the NMDA receptor antagonist of the first portion is no more than about 50 mg, 75 mg, 100 mg, 125 mg, 150 mg, 175 mg, 200 mg, 225 mg, 250 mg, 275 mg or 300 mg.

In one embodiment, the NMDA receptor antagonist (e.g., ketamine or a metabolite or analog thereof) of the first portion is at least about 10%, or 20%, or 30%, or 40%, of 50%, or 60%, or 70%, or 80%, or 90% of a therapeutically effective amount. In one embodiment, the NMDA receptor antagonist of the first portion is at most about 10%, or 20%, or 30%, or 40%, of 50%, or 60%, or 70%, or 80%, or 90% of a therapeutically effective amount. In one embodiment, the NMDA receptor antagonist of the second portion is at least about 10%, or 20%, or 30%, or 40%, of 50%, or 60%, or 70%, or 80%, or 90% of a therapeutically effective amount. In one embodiment, the NMDA receptor antagonist of the second portion is at most about 10%, or 20%, or 30%, or 40%, of 50%, or 60%, or 70%, or 80%, or 90% of a therapeutically effective amount.

In one embodiment, the first portion of the NMDA receptor antagonist (e.g., ketamine or a metabolite or analog thereof) constitutes at least about 10% of the total NMDA receptor antagonist in the composition. Alternatively, the first portion of the NMDA receptor antagonist constitutes at least about 20%, or 30%, or 40%, or 50%, or 60%, or 70%, or 80%, or 90% of the total NMDA receptor antagonist. In some embodiments, however, the first portion of the NMDA receptor antagonist can be less than about 20%, or 30%, or 40%, or 50%, or 60%, or 70%, or 80%, or 90% of the total NMDA receptor antagonist. In a particular embodiment, the first portion constitutes from about 40% to about 60%, or alternatively from about 45% to about 55% of the total NMDA receptor antagonist.

In one embodiment, the total amount of the NMDA receptor antagonist (e.g., ketamine or a metabolite or analog thereof) in the composition is less than about 50 mg, or 60 mg, or 70 mg, or 80 mg, or 90 mg, or 100 mg, or 120 mg, or 140 mg, or 150 mg, or 160 mg, or 165 mg, or 170 mg, or 180 mg, or 190 mg, 200 mg, 210 mg, 220 mg, 230 mg, 240 mg, 250 mg, 260 mg, 270 mg, 280 mg, 290 mg, or 300 mg. In another embodiment, the total amount of the NMDA receptor antagonist in the composition is greater than about 10 mg, 20 mg, or 30 mg, or 40 g, or 50 mg, or 60 mg, or 70 mg, or 80 mg, or 90 mg, or 100 mg, or 110 mg, or 120 mg, or 130 mg, or 140 mg, or 150 mg.

It is further contemplated that the addition of aspirin (or more generally an NSAID) can further enhance the bioavailability and/or efficacy of the NMDA receptor antagonist (e.g., ketamine or a metabolite or analog thereof). In one embodiment, aspirin (or NSAID) is added to the first portion of the NMDA receptor antagonist which disintegrates or dissolves intraorally providing rapid release of the NMDA receptor antagonist of the first portion. In some embodiments, the aspirin in the first portion is at least about 40 mg. In some embodiments, the aspirin in the first portion is at least about 80 mg, 160 mg, 240 mg or 320 mg.

In one embodiment, aspirin is added to the second portion of the NMDA receptor antagonist which is substantially more difficult than the first portion to disintegrate or dissolve intraorally but is ingestible and releasable in the gastrointestinal tract of the subject. In some embodiments, the aspirin in the second portion is at least about 40 mg. In some embodiments, the aspirin in the second portion is at least about 80 mg, 160 mg, 240 mg or 320 mg.

Another embodiment of the invention provides a process of preparing the disclosed compositions. In some embodiments, the process comprises forming a core composition (e.g., a compressed core tablet, or a core capsule) then coating the core composition with one or more layers to form a dual-release solid oral dosage form. Preparation of each portion is further described below.

### 1. First Portion of NMDA Receptor Antagonist for Intraoral Release

Methods of preparing a composition suitable for intraoral release are known in the art. In one embodiment, the first portion further includes a film-coating agent, an excipient, a binder, a lubricant, or a plasticizer.

In one embodiment, the first portion comprising an NMDA receptor antagonist (e.g. ketamine) disintegrates or dissolves intraorally within about 10 minutes. In other embodiments, the first portion disintegrates or dissolves intraorally within about 9 minutes, or about 8, or about 7, or about 6, or about 5, or about 4, or about 3 or about 2 minutes, or alternatively about 60 seconds, or about 50, or about 40, or about 30, or about 20, or about 10, or about 5 seconds.

In some embodiments, the first portion comprising an NMDA receptor antagonist (e.g. ketamine) is chewable. In some embodiments, the first portion is in the form of molded triturate.

In one embodiment, the first portion comprising an NMDA receptor antagonist (e.g. ketamine) further includes an agent that promotes the oral or buccal absorption of the ketamine. Non-limiting examples of such agents include bile acid salts, sodium lauryl sulfate, lysalbinic acid, salicylic acid, 5-methoxy salicylic acid, 3,4-dihydroxy phenyl acetic acid (DOPAC) and homovanillic acid and their sodium salts thereof. Other hydroxyaryl acids, such as 1-hydroxy-2-naphthoic acid, naphthoresorcyclic acid, ferulic acid, caffeic acid, resorcylic acid and gentisic acid, have similar effects.

The amount of hydroxyaryl or hydroxyaralkyl acid or salt, amide or ester derivatives thereof forms may vary over a wide range; in general, the identity and the amount of the hydroxyaryl or hydroxyaralkyl acids or salt, amide or ester thereof is used in connection with the drug in order to be effective in enhancing the absorption rate of the drug into the bloodstream.

In another embodiment, the first portion comprising an NMDA receptor antagonist (e.g. ketamine) further includes a disintegrant. Non-limiting examples of disintegrants include crospovidone, crystalline cellulose, hydroxypropylcellulose with a low degree of substitution, croscarmellose sodium, carmellose calcium, carboxystarch sodium, carboxymethyl starch sodium, potato starch, wheat starch, corn starch, rice starch, partly pregelatinized starch, and hydroxypropyl starch. One or two or more of these can be used together. Coating with a disintegrant also contributes to improvement of compression moldability.

In some embodiments, the first portion comprising an NMDA receptor antagonist (e.g. ketamine) comprises a plasticizer. Non limiting examples of plasticizers include dibutyl sebacate (DBS), dimethyl sebacate (DMS), dioctyl sebacate (DOS), acetyl tributyl citrate, or tributyl citrate.

In another embodiment, the first portion comprising an NMDA receptor antagonist (e.g. ketamine) includes a binder such as microcrystalline cellulose. Inclusion of a binder also contributes to improvement of compression moldability.

### 2. First Portion of GRIN2A Modulator for Intraoral Release

Methods of preparing a composition suitable for intraoral release are known in the art. In one embodiment, the first portion further includes a film-coating agent, an excipient, a binder, a lubricant, or a plasticizer.

In one embodiment, the first portion comprising a GRIN2A modulator (e.g., aspirin) disintegrates or dissolves intraorally within about 10 minutes. In other embodiments, the first portion disintegrates or dissolves intraorally within about 9 minutes, or about 8, or about 7, or about 6, or about 5, or about 4, or about 3 or about 2 minutes, or alternatively about 60 seconds, or about 50, or about 40, or about 30, or about 20, or about 10, or about 5 seconds.

In some embodiments, the first portion comprising a GRIN2A modulator (e.g., aspirin) is chewable. In some embodiments, the first portion is in the form of molded triturate.

In some embodiments, the first portion comprising a GRIN2A modulator (e.g., aspirin) comprises a disintegrant. Non-limiting examples of disintegrants include crospovidone, crystalline cellulose, hydroxypropylcellulose with a low degree of substitution, croscarmellose sodium, carmellose calcium, carboxystarch sodium, carboxymethyl starch sodium, potato starch, wheat starch, corn starch, rice starch, partly pregelatinized starch, and hydroxypropyl starch. One or two or more of these can be used together. Coating with a disintegrant also contributes to improvement of compression moldability.

In some embodiments, the first portion comprising a GRIN2A modulator (e.g., aspirin) comprises a plasticizer. Non limiting examples of plasticizers include dibutyl sebacate (DBS), dimethyl sebacate (DMS), dioctyl sebacate (DOS), acetyl tributyl citrate, or tributyl citrate.

In another embodiment, the first portion comprising a GRIN2A modulator (e.g., aspirin) includes a binder such as microcrystalline cellulose. Inclusion of a binder also contributes to improvement of compression moldability.

### 3. Second Portion of NMDA Receptor Antagonist and/or GRIN2A Modulator, for GI Release

The second portion of the composition can be prepared with methods known in the art for a typical oral dosage form suitable for GI absorption. Like the first portion, the second portion can also include a film-coating agent, an excipient, a binder, a lubricant, and/or a plasticizer. In some embodiments, the second portion is a compressed tablet which forms a core. In some of such embodiments, the core composition comprises ketamine and aspirin. In some of such embodiments, the core composition comprises ketamine. In some of such embodiments, the core composition comprises aspirin.

Compared to the first portion, the second portion is substantially more difficult to disintegrate or dissolve intraorally. This can be achieved chemically or physically. For instance, the second portion can be physically harder. In one embodiment, the second portion is compressed. In another embodiment, the second portion has a hardness that is at least about 10 kilopascal (kp), or alternatively about 11, or 12, or 13, or 14, or 15, or 20, or 25 or 30 or 40 or 50 kp.

Hardness can be assessed by means commonly used in the art, for example, using commercially available hardness testers that are routinely used for assessing the hardness of pharmaceutical dosage forms.

In some embodiments, the second portion further comprises a pharmaceutically acceptable flavoring agent not present in the first portion. The flavoring agent provides a flavor that alerts the patients that this portion should not be chewed and needs to be swallowed so as to increase patient compliance.

In one embodiment, the total NMDA receptor antagonist (e.g. ketamine) in the second portion constitutes at least about 10% of the total NMDA receptor antagonist (e.g. ketamine) in the tablet. Alternatively, the NMDA receptor antagonist (e.g. ketamine) in the second portion constitutes at least about 20%, or 30%, or 40%, or 50%, or 60%, or 70%, or 80%, or 90% of the total NMDA receptor antagonist. In some embodiments, however, the NMDA receptor antagonist in the second portion can be less than about 20%, or 30%, or 40%, or 50%, or 60%, or 70%, or 80%, or 90% of the total NMDA receptor antagonist. In a particular embodiment, the NMDA receptor antagonist in the second portion constitutes from about 40% to about 60%, or alternatively from about 45% to about 55% of the total NMDA receptor antagonist. In one embodiment, the ratio of the NMDA receptor antagonist between the first portion and the second portion is about 1:1. Alternatively, the ratio is at least about 1:4, or 1:3, or 1:2 or 1:1.5, or is no more than about 4:1, 3:1, 2:1 or 1.5:1.

In one embodiment, the total GRIN2A modulator (e.g. aspirin) in the second portion constitutes at least about 10% of the total GRIN2A modulator (e.g., aspirin) in the tablet. Alternatively, the GRIN2A modulator (e.g., aspirin) in the second portion constitutes at least about 20%, or 30%, or 40%, or 50%, or 60%, or 70%, or 80%, or 90% of the total GRIN2A modulator. In some embodiments, however, the GRIN2A modulator in the second portion can be less than about 20%, or 30%, or 40%, or 50%, or 60%, or 70%, or 80%, or 90% of the total GRIN2A modulator. In a particular embodiment, the GRIN2A modulator in the second portion constitutes from about 40% to about 60%, or alternatively from about 45% to about 55% of the total GRIN2A modulator. In one embodiment, the ratio of the GRIN2A modulator between the first portion and the second portion is about 1:1. Alternatively, the ratio is at least about 1:4, or 1:3, or 1:2 or 1:1.5, or is no more than about 4:1, 3:1, 2:1 or 1.5:1.

The pharmaceutical composition of the present disclosure can be in the form of a tablet or capsule. When in the form of a tablet, the second portion, in one embodiment, is enclosed within the first portion, or alternatively, partially exposed.

When the composition is in the form of a tablet, the tablet can include an outer portion and an inter portion, with the outer portion containing the first portion and the inner portion containing the second portion.

In one embodiment, the outer portion is formulated to dissolve in the oral cavity of a subject and to release the NMDA receptor antagonist and/or the GRIN2A modulator in the first portion across the oral mucosa of the subject. In one embodiment, the inner portion is harder than the outer portion and is formulated for dissolving in stomach, intestines, or distally in the gastrointestinal tract of the subject.

In one embodiment, the inner portion comprises a texture on the surface that is recognizable by the tongue of a subject. In another embodiment, the outer portion comprises a water soluble sugar or sugar substitute. In another embodiment, the outer portion is surrounded by a thin shell to allow encapsulation of liquid, powder or gel in the outer portion.

In one embodiment, the outer potion is flavored or sweetened. In one embodiment, the tablet optionally comprises an intermediate layer between the outer portions and the inner portion. In one embodiment, the intermediate layer comprises an enteric coating. In one embodiment, the inner portion/core composition is formulated to absorb a biting shock and not break a tooth. In another embodiment, the tablet comprises a core layer of ketamine which breaks down in the mouth, but this layer has particles within it that do not completely break down in the mouth and stay as full particles, such that there is partial intraoral release and, when the particles are swallowed, partial gastrointestinal release.

### 4. Dual Release NMDA Receptor Antagonist Composition

In one embodiment, provided is a pharmaceutical composition comprising a NMDA receptor antagonist (e.g. ketamine), wherein, upon oral administration to a subject, a first portion disintegrates or dissolves intraorally providing rapid release of ketamine, and a second portion is substantially more difficult than the first portion to disintegrate or dissolve intraorally but is ingestible and releases ketamine in the gastrointestinal tract of the subject.

In some embodiments the pharmaceutical composition comprises:
(i) a core composition comprising ketamine; and
(ii) a coat comprising ketamine and enclosing the core composition.

In such embodiments, the coat (ii) forms the first portion which disintegrates or dissolves intraorally providing rapid release of ketamine. In such embodiments, the core composition (i) forms the second portion that is substantially more difficult than the first portion to disintegrate or dissolve intraorally but is ingestible and releases ketamine in the gastrointestinal tract.

In some embodiments, the core composition (i) comprises about 1% w/w to about 15% w/w of ketamine. In some embodiments, the core composition (i) comprises about 60% w/w to about 80% or to about 95%) w/w of a diluent (e.g., microcrystalline cellulose). In some embodiments, the core composition (i) comprises about 10% w/w to about 20% w/w of a binder (e.g., pregelatinized starch, povidone). In some embodiments, the core composition (i) comprises about 0.4% w/w (or about 1%) to about 4% w/w of a disintegrant (e.g., croscarmellose sodium, crospovidone). In some embodiments, the core composition (i) comprises about 0.1% w/w to about 0.5% w/w, or 0.5% w/w to about 1.5% w/w, of a glidant (e.g., silicon dioxide, talc). In some embodiments, the core composition (i) comprises about 0.1% w/w to about 0.5% w/w of a lubricant (e.g., stearic acid, sodium stearyl fumarate). In such embodiments, the % w/w is based on the total weight of the core composition.

In some embodiments, the core composition (i) comprises ketamine, microcrystalline cellulose, croscarmellose sodium, colloidal silicon dioxide, and stearic acid. In some embodiments, the core composition is a compressed tablet.

In some embodiments, the core composition comprises about 3% w/w to about 10% w/w of ketamine, wherein % w/w refers to percent by weight of the total weight of the core composition. In some embodiments, the core composition comprises about 3% w/w to about 5% w/w, or about 4% w/w of ketamine, wherein % w/w refers to percent by weight of the total weight of the core composition.

In some of such embodiments, the core composition's ketamine is about 10%, about 20%, about 30%, about 40%, about 50%, about 60%, about 70%, about 80%, about 90%, or about 95% of the total ketamine present in the pharmaceutical composition. In some of such embodiments, the core composition's ketamine is released substantially in the GI tract.

In some embodiments, the coat (ii) comprising ketamine is substantially free of an alcohol. In some embodiments, the coat (ii) comprising ketamine further comprises a binder, a plasticizer, and a disintegrant.

In some embodiments, the coat (ii) comprises about 1% w/w to about 10% w/w of ketamine. In some embodiments, the coat (ii) comprises about 1% w/w about 5% w/w of a disintegrant (e.g., hydroxypropyl methylcellulose, hydroxypropyl cellulose, methyl cellulose,, or any combination thereof). In some embodiments, the coat (ii) optionally comprises from about 0.1% w/w to about 0. 5% w/w of polyethylene glycol or triacetin. In some embodiments, the coat (ii) comprises about 1% w/w to about 5% w/w of a binder (e.g., microcrystalline cellulose). In some embodiments, the coat (ii) comprises about 0.01% w/w to about 0.05% w/w sweetener (e.g., sucralose, aspartame). In some embodiments, the coat (ii) comprises about 0.01% w/w to about 0.1% w/w flavoring agent (e.g., grape flavoring, mixed berry flavoring). In some embodiments, the coat (ii) comprises about 0.01% w/w to about 0.05% w/w pigment. In such embodiments, the % w/w refers to the weight of the coat added onto the core composition (i).

In some embodiments, the coat (ii) comprises about 25% w/w to about 45% w/w of ketamine. In some embodiments, the coat (ii) comprises about 20% w/w about 40% w/w of a binder (e.g., hydroxypropyl methylcellulose, hydroxypropyl cellulose, methyl cellulose,, or any combination thereof). In some embodiments, the coat (ii) optionally comprises from about 1% w/w to about 5% w/w of polyethylene glycol or triacetin. In some embodiments, the coat (ii) comprises about 25% w/w to about 45% w/w of a disintegrant (e.g., microcrystalline cellulose). In some embodiments, the coat (ii) comprises about 0.05% w/w to about 0.5% w/w sweetener (e.g., sucralose, aspartame). In some embodiments, the coat (ii) comprises about 0.1% w/w to about 1% w/w flavoring agent (e.g., grape flavoring, mixed berry flavoring). In some embodiments, the coat (ii) comprises about 0.1% w/w to about 0.5% w/w pigment. In such embodiments, the % w/w refers to the weight of the coat added onto the core composition (i).

In some embodiments, the binder comprises hydroxypropyl methylcellulose (HPMC), ethyl cellulose, methyl cellulose, hydroxypropyl cellulose, or any combination thereof. In some embodiments, the binder comprises hydroxypropyl methylcellulose. In some embodiments, the binder comprises methyl cellulose, and hydroxypropyl cellulose.

In some embodiments, the plasticizer is dibutyl sebacate (DBS), dimethyl sebacate (DMS), dioctyl sebacate (DOS), triethyl citrate, triacetin, acetyl tributyl citrate, tributyl citrate, triethyl citrate, polyethylene glycol, or any combination thereof. In some embodiments, the plasticizer is dibutyl sebacate (DBS). In some embodiments, the plasticizer is triacetin. In some embodiments, the plasticizer is polyethylene glycol and DBS. In some embodiments, the plasticizer is polyethylene glycol and triacetin.

In some embodiments, the disintegrant is microcrystalline cellulose (MCC). Other disintegrants include and are not limited to starch, pre-gelatinized starch, sodium starch glycolate, and croscarmellose sodium.

In some embodiments, the coat comprising ketamine is prepared by spray coating a substrate with an aqueous solution comprising ketamine, a binder, and a disintegrant. In some embodiments, the spray coating solution further comprises one or more flavoring agents and one or more pigments. In some of such embodiments, the substrate that is spray coated is the core composition (i).

In some embodiments, the composition optionally further comprises sweeteners, pigments, and flavoring agents in the core composition, and/or the coat comprising ketamine. In some of such embodiments, the flavoring agent of the core composition may be the same as the flavoring agent in the coat, or may be different from the flavoring agent of the coat.

In some embodiments, up to 50% of the total ketamine in the composition is released intraorally. In some embodiments, up to 10%, up to 20%, up to 30%, up to 40%, up to 50%, up to 60%, up to 70%, up to 80%, up to 90%, or up to 95%, of the total ketamine in the composition is released intraorally. In some embodiments, at least 5%, at least 10%, at least 20%, at least 30%, at least 40%, at least 50%, at least 60%, at least 70%, at least 80%, at least 90%, or at least 95%, of the total ketamine in the composition is released in the GI tract.

Provided is a dual release pharmaceutical composition comprising ketamine, wherein, upon oral administration to a subject, a first portion disintegrates or dissolves intraorally providing rapid release of ketamine, and a second portion is substantially more difficult than the first portion to disintegrate or dissolve intraorally but is ingestible and releases ketamine in the gastrointestinal tract of the subject,
wherein the composition comprises:
(i) a core composition comprising:
   (i-a) about 3% w/w to about 10% w/w ketamine;
   (i-b) about 60% w/w to about 80% w/w microcrystalline cellulose;
   (i-c) about 10% w/w to about 20% w/w of pregelatinized starch;
   (i-d) about 1% w/w/to about 4% w/w croscarmellose sodium;
   (i-f) about 0.5% w/w to about 1.5% w/w silicon dioxide; and
   (i-g) about 0.1% w/w to about 0.5% w/w stearic acid;
   wherein the % w/w is based on the total weight of the core composition;
(ii) a coat comprising ketamine and enclosing the core composition, and providing about 10% solid weight gain to the core composition (i), wherein the coat comprises
   (ii-a) about 1% w/w about 5% w/w ketamine;
   (ii-b) about 1% w/w about 5% w/w hydroxypropyl methylcellulose;
   (ii-c) about 0.1% w/w to about 0.5% w/w polyethylene glycol;
   (ii-d) about 1% w/w to about 5% w/w microcrystalline cellulose;
   (ii-e) about 0.01% w/w to about 0.05% w/w sweetener;
   (ii-f) about 0.01% w/w to about 0.1% w/w flavoring agent; and
   (ii-g) about 0.01% w/w to about 0.05% w/w pigment;
   wherein the % w/w is based on the weight of the coat.

In some embodiments, the composition comprises:
(i) a core composition comprising:
   (i-a) about 3% w/w to about 10% w/w ketamine;
   (i-b) about 60% w/w to about 95% w/w microcrystalline cellulose;
   (i-c) about 0.4% w/w/to about 4% w/w croscarmellose sodium;
   (i-d) about 0.1% w/w to about 0.5% w/w silicon dioxide; and
   (i-e) about 0.1% w/w to about 0.5% w/w stearic acid;
   wherein the % w/w is based on the total weight of the core composition;
(ii) a coat comprising ketamine and enclosing the core composition, and providing about 10%-20% solid weight gain to the core composition (i), wherein the coat comprises
   (ii-a) about 25% w/w about 45% w/w ketamine;
   (ii-b) about 20% w/w about 40% w/w hydroxypropyl methylcellulose;
   (ii-c) about 1% w/w to about 5% w/w polyethylene glycol;
   (ii-d) about 25% w/w to about 45% w/w microcrystalline cellulose;
   (ii-e) about 0.05% w/w to about 0.5% w/w sweetener;
   (ii-f) about 0.1% w/w to about 1% w/w flavoring agent; and
   (ii-g) about 0.1% w/w to about 0.5% w/w pigment;
wherein the % w/w is based on the weight of the coat.

In such embodiments, the coat (ii) forms the first portion which disintegrates or dissolves intraorally providing rapid release of ketamine, and the core composition (i) forms the second portion that is substantially more difficult than the first portion to disintegrate or dissolve intraorally but is ingestible and releases ketamine in the gastrointestinal tract.

In some embodiments, a dual release ketamine pharmaceutical composition comprises ketamine in a core composition, formulated for GI release, and a coating on the core composition comprising ketamine wherein the coating on the core composition allows for intraoral release of ketamine. One or more coats comprising ketamine may be present on the core composition. In some embodiments, a seal coat may be present as the final coat. In some embodiments, a seal coat is present between the core composition and the coats comprising ketamine that is formulated for intraoral release. In some embodiments, a seal coat is present between the core composition and the coats comprising ketamine that is formulated for intraoral release and also as a final coat.

The pharmaceutical composition of the above embodiments can further include a third portion that comprises an effective amount of ketamine. In one embodiment, the third portion is in the form of controlled release. In another embodiment, the third portion further comprises enteric coating. In yet another embodiment, the third portion is enclosed in the first portion and/or the second portion.

### 5. Dual Release GRIN2A Modulator Composition

In some embodiments, provided is a pharmaceutical composition comprising aspirin, wherein, upon oral administration to a subject, a first portion disintegrates or dissolves intraorally providing rapid release of aspirin, and a second portion is substantially more difficult than the first portion to disintegrate or dissolve intraorally but is ingestible and releases aspirin in the gastrointestinal tract of the subject.

In some embodiments, the composition comprises:
(i) a core composition comprising aspirin; and
(ii) a coat comprising aspirin and enclosing the core composition.

In such embodiments, the coat (ii) forms the first portion which disintegrates or dissolves intraorally providing rapid release of aspirin. In such embodiments, the core composition (i) forms the second portion that is substantially more difficult than the first portion to disintegrate or dissolve intraorally but is ingestible and releases aspirin in the gastrointestinal tract.

In some embodiments, the core composition (i) comprises about 30% w/w to about 40% w/w aspirin. In some embodiments, the core composition (i) comprises about 40% w/w to about 80% w/w of a diluent (e.g., microcrystalline cellulose). In some embodiments, the core composition (i) comprises about 10% w/w to about 20% w/w of a binder (e.g., pregelatinized starch, povidone). In some embodiments, the core composition (i) comprises about 1% w/w to about 10% w/w of a pH modifier (e.g., citric acid). In some embodiments, the core composition (i) comprises about 0.4% w/w to about 4% w/w of a disintegrant (e.g., croscarmellose sodium, crospovidone). In some embodiments, the core composition (i) comprises about 0.1% w/w to about 0.5% w/w of a glidant (e.g., silicon dioxide, talc). In some embodiments, the core composition (i) comprises about 0.1% w/w to about 0.5% w/w of a lubricant (e.g., stearic acid, sodium stearyl fumarate). In such embodiments, the % w/w is based on the total weight of the core composition (i). In some embodiments, the core composition (i) is a compressed tablet.

In some embodiments, the core composition (i) comprises aspirin, microcrystalline cellulose, croscarmellose sodium, colloidal silicon dioxide, and stearic acid.

In some embodiments, the core composition (i) comprises about 30% w/w to about 40% w/w of aspirin, wherein % w/w refers to percent by weight of the total weight of the core composition. In some embodiments, the core composition (i) comprises about 30% w/w to about 35% w/w, or about 32% w/w of aspirin, wherein % w/w refers to percent by weight of the total weight of the core composition.

In some embodiments, the coat (ii) comprising aspirin is substantially free of water.

In some embodiments, the coat (ii) comprising aspirin further comprises a binder, a plasticizer, a disintegrant, and a pH modifier.

In some embodiments, the coat (ii) comprises about 20% w/w about 40% w/w aspirin. In some embodiments, the coat (ii) comprises about 5% w/w about 15% w/w of a binder (e.g., hydroxypropyl methylcellulose, hydroxypropyl cellulose, methyl cellulose). In some embodiments, the coat (ii) comprises about 15% w/w to about 30% w/w of a disintegrant (e.g., microcrystalline cellulose). In some embodiments, the coat (ii) comprises about 10% w/w to about25% w/w of a plasticizer (e.g., dibutyl sebacate, tributyl citrate). In some embodiments, the coat (ii) comprises about 15% w/w to about 30% w/w of a pH modifier (e.g., citric acid). In some embodiments, the coat (ii) comprises about 0.1% w/w to about 0.5% w/w sweetener (e.g. sucralose, aspartame). In some embodiments, the coat (ii) comprises about 0.1% w/w to about 1.5% w/w flavoring agent (e.g. grape flavoring, mixed berry flavoring). In some embodiments, the coat (ii) comprises about 0.1% w/w to about 0.8% w/w pigment. In such embodiments, the % w/w is based on the weight of the coat.

In some embodiments, the binder is hydroxypropyl cellulose (HPC), hydroxypropyl methylcellulose (HPMC), methyl cellulose, or polyvinylpyrrolidone (PVP), or a combination thereof.

In some embodiments, the plasticizer is dibutyl sebacate (DBS), dimethyl sebacate (DMS), dioctyl sebacate (DOS), triacetin, acetyl tributyl citrate, tributyl citrate, triethyl citrate, polyethylene glycol, or any combination thereof. In some embodiments, the plasticizer is DBS. In some embodiments, the plasticizer is tribuyl citrate. In some embodiments, the plasticizer is polyethylene glycol and DBS. In some embodiments, the plasticizer is polyethylene glycol and triacetin.

In some embodiments, the disintegrant is microcrystalline cellulose (MCC). Other disintegrants include and are not limited to starch, pre-gelatinized starch, sodium starch glycolate, and croscarmellose sodium.

In some embodiments, the pH modifier is citric acid or a salt thereof.

In some embodiments, the coat (ii) comprising aspirin is prepared by spray coating a core composition with a solution comprising aspirin, a binder, a plasticizer, a disintegrant, a pH modifier, and an alcohol. In some of such embodiments, the spray solution further comprises one or more flavoring agents, one or more sweeteners, and one or more pigments.

In some embodiments, the alcohol used for the spray solution is isopropyl alcohol (IPA), or ethanol.

In some embodiments, the composition optionally further comprises sweeteners, pigments, and flavoring agents in the core composition, and/or the coat comprising aspirin. In some of such embodiments, the flavoring agents in the core composition (i) may be the same as the coat (ii), or may be different from the flavoring agents in the coat (ii).

In some embodiments, up to 50% of the total aspirin in the composition is released intraorally. In some embodiments, up to 10%, up to 20%, up to 30%, up to 40%, up to 50%, up to 60%, up to 70%, up to 80%, up to 90%, or up to 95%, of the total aspirin in the composition is released intraorally. In some embodiments, at least 5%, at least 10%, at least 20%, at least 30%, at least 40%, at least 50%, at least 60%, at least 70%, at least 80%, at least 90%, or at least 95%, of the total aspirin in the composition is released in the GI tract.

Provided is a dual release pharmaceutical composition comprising aspirin, wherein, upon oral administration to a subject, a first portion disintegrates or dissolves intraorally providing rapid release of aspirin, and a second portion is substantially more difficult than the first portion to disintegrate or dissolve intraorally but is ingestible and releases aspirin and ketamine in the gastrointestinal tract of the subject,
wherein the composition comprises:
(i) a core composition comprising:
   (i-a) about 30% w/w to about 40% w/w aspirin;
   (i-b) about 40% w/w to about 60% w/w microcrystalline cellulose;
   (i-c) about 10% w/w to about 20% w/w of pregelatinized starch;
   (i-d) about 1% w/w to about 10% w/w citric acid;
   (i-e) about 1% w/w to about 4% w/w croscarmellose sodium;
   (i-f) about 0.5% w/w to about 1.5% w/w silicon dioxide; and
   (i-h) about 0.1% w/w to about 0.5% w/w stearic acid;
   wherein the % w/w is based on the total weight of the core composition;
(ii) a coat comprising aspirin and enclosing the core composition, and providing about 15% solid weight gain to the core composition (i), wherein the coat comprises
   (ii-a) about 1% w/w about 7% w/w aspirin;
   (ii-b) about 1% w/w about 5% w/w hydroxypropyl cellulose;
   (ii-c) about 2% w/w to about 6% w/w microcrystalline cellulose;
   (ii-d) about 1.5% w/w to about 4% w/w of dibutyl sebacate;
   (ii-e) about 2% w/w to about 5% w/w citric acid;
   (ii-f) about 0.01% w/w to about 0.05% w/w sweetener;
   (ii-g) about 0.01% w/w to about 0.2% w/w flavoring agent; and
   (ii-h) about 0.01% w/w to about 0.1% w/w pigment;
   wherein the % w/w is based on the weight of the coat.

In some embodiments, the composition comprises:
(i) a core composition comprising:
   (i-a) about 30% w/w to about 40% w/w aspirin;
   (i-b) about 40% w/w to about 80% w/w microcrystalline cellulose;
   (i-c) about 0.4% w/w to about 4% w/w croscarmellose sodium;
   (i-d) about 0.1% w/w to about 0.5% w/w silicon dioxide; and
   (i-e) about 0.1% w/w to about 0.5% w/w stearic acid;
   wherein the % w/w is based on the total weight of the core composition;
(ii) a coat comprising aspirin and enclosing the core composition, and providing about 120-140% solid weight gain to the core composition (i), wherein the coat comprises
   (ii-a) about 20% w/w about 40% w/w aspirin;
   (ii-b) about 5% w/w about 15% w/w hydroxypropyl cellulose;
   (ii-c) about 15% w/w to about 30% w/w microcrystalline cellulose;
   (ii-d) about 10% w/w to about 25% w/w of dibutyl sebacate;
   (ii-e) about 15% w/w to about 30% w/w citric acid;
   (ii-f) about 0.1% w/w to about 0.5% w/w sweetener;
   (ii-g) about 0.1% w/w to about 1.5% w/w flavoring agent; and
   (ii-h) about 0.1% w/w to about 0.8% w/w pigment;
wherein the % w/w is based on the weight of the coat.

In such embodiments, the coat (ii) forms the first portion which disintegrates or dissolves intraorally providing rapid release of aspirin, and the core composition (i) forms the second portion that is substantially more difficult than the first portion to disintegrate or dissolve intraorally but is ingestible and releases aspirin in the gastrointestinal tract.

In some embodiments, a dual release aspirin pharmaceutical composition comprises aspirin in a core composition, formulated for GI release, and a coating on the core composition comprising aspirin wherein the coating on the core composition allows for intraoral release of aspirin. One or more coats comprising aspirin may be present on the core composition. In some embodiments, a seal coat may be present as the final coat. In some embodiments, a seal coat is present between the core composition and the coats comprising aspirin that is formulated for intraoral release. In some embodiments, a seal coat is present between the core composition and the coats comprising aspirin that is formulated for intraoral release and also as a final coat.

The pharmaceutical composition of the above embodiments can further include a third portion that comprises an effective amount of aspirin. In one embodiment, the third portion is in the form of controlled release. In another embodiment, the third portion further comprises enteric coating. In yet another embodiment, the third portion is enclosed in the first portion and/or the second portion.

### 5. Combination Dosage Form of NMDA Receptor Antagonist and GRIN2A Modulator

Certain desired effects of an NMDA receptor antagonist (e.g., ketamine or a metabolite or analog thereof), such as pain reduction, suppression of depression, reduction of fatigue, can be further enhanced by co-administration with aspirin. The co-administration of aspirin, or another GRIN2A modulator, is also associated with a reduction of the undesired effects of the NMDA receptor antagonist, including its sedation effect and addiction potential.

In accordance with one embodiment of the disclosure, provided is a pharmaceutical composition comprising an NMDA receptor antagonist (e.g., ketamine or a metabolite or analog thereof) and a glutamate ionotropic receptor NMDA type subunit 2A (GRIN2A) modulator. In some embodiments, the GRIN2A modulator is a GRIN2A activator. Non-limiting examples include aspirin, nicotine, propofol, melatonin and GQ1b.

In some embodiments, the NMDA receptor antagonist in the composition is provided as two portions, with the first portion comprising a first amount of the NMDA receptor antagonist, and a second portion comprising a second amount of the NMDA receptor antagonist, wherein, upon oral administration to a subject, the first portion disintegrates or dissolves intraorally providing rapid release of the NMDA receptor antagonist of the first portion, and the second portion is substantially more difficult than the first portion to disintegrate or dissolve intraorally but is ingestible and releasable in the gastrointestinal tract of the subject.

In some embodiments, the GRIN2A modulator is formulated together with the first portion of the NMDA receptor antagonist. In some embodiments, the GRIN2A modulator is formulated together with the second portion of the NMDA receptor antagonist. In some embodiments, the GRIN2A modulator is formulated together with each of the first portion and the second portion of the NMDA receptor antagonist.

In another embodiment, the present disclosure provides a pharmaceutical composition comprising an NMDA receptor antagonist and aspirin. Either or both of the NMDA receptor antagonist and aspirin can be formulated for both intraoral and GI administration, as described throughout.

Provided is a pharmaceutical composition comprising ketamine and aspirin, wherein, upon oral administration to a subject, a first portion disintegrates or dissolves intraorally providing rapid release of aspirin and ketamine, and a second portion is substantially more difficult than the first portion to disintegrate or dissolve intraorally but is ingestible and releases aspirin and ketamine in the gastrointestinal tract of the subject.

In some embodiments, the composition comprises:
(i) a core composition comprising aspirin and ketamine;
(ii) a first coat comprising ketamine and enclosing the core composition;
(iii) a second coat comprising a seal coat and enclosing the core composition coated with the first coat; and
(iv) a third coat comprising aspirin and enclosing the core composition coated with the first coat and the second coat.

In such embodiments, the coats (ii)-(iv) form the first portion which disintegrates or dissolves intraorally providing rapid release of ketamine and aspirin. In such embodiments, the core composition (i) forms the second portion that is substantially more difficult than the first portion to disintegrate or dissolve intraorally but is ingestible and releases ketamine and aspirin in the gastrointestinal tract.

In some embodiments, the composition comprises:
(i) a core composition comprising aspirin and ketamine;
(ii) a first coat comprising aspirin and enclosing the core composition;
(iii) a second coat comprising a seal coat and enclosing the core composition coated with the first coat; and
(iv) a third coat comprising ketamine and enclosing the core composition coated with the first coat and the second coat.

In such embodiments, the coats (ii)-(iv) form the first portion which disintegrates or dissolves intraorally providing rapid release of ketamine and aspirin. In such embodiments, the core composition (i) forms the second portion that is substantially more difficult than the first portion to disintegrate or dissolve intraorally but is ingestible and releases ketamine and aspirin in the gastrointestinal tract.

In some embodiments, the core composition (i) comprises about 3% w/w to about 35% w/w ketamine. In some embodiments, the core composition (i) comprises about 30% w/w to about 50% w/w aspirin. In some embodiments, the core composition (i) comprises about 15% w/w to about 60% w/w of a diluent (e.g., microcrystalline cellulose). In some embodiments, the core composition (i) comprises about 5% w/w to about 20% w/w of a binder (e.g., pregelatinized starch, povidone). In some embodiments, the core composition (i) comprises about 0.5% w/w to about 5% w/w of an a pH modifier (e.g., citric acid). In some embodiments, the core composition (i) comprises about 0.2% w/w/to about 4% w/w of a disintegrant (e.g., croscarmellose sodium, crospovidone). In some embodiments, the core composition (i) comprises about 0.1% w/w to about 1.5% w/w of a glidant (e.g., silicon dioxide, talc). In some embodiments, the core composition (i) comprises about 0.01% w/w to about 0.5% w/w of a lubricant (e.g., stearic acid, sodium stearyl fumarate). In such embodiments, the % w/w is based on the total weight of the core composition;

In some embodiments, the core composition comprises aspirin, ketamine, microcrystalline cellulose, pregelatinized starch, citric acid anhydrous, croscarmellose sodium, colloidal silicon dioxide, and stearic acid.

In some embodiments, the core composition (i) comprises about 3% w/w to about 35% w/w of ketamine and about 30% w/w to about 50% w/w of aspirin, wherein % w/w refers to percent by weight of the total weight of the core composition. In some embodiments, the core composition (i) comprises about 3% w/w to about 10% w/w of ketamine and about 35% w/w to about 40% w/w of aspirin, or about 5% w/w of ketamine and about 36% w/w of aspirin.

The core tablet (i) may be coated with one or more ketamine coats and one or more aspirin coats in any order. For instance, in one embodiment, coat (i) comprises aspirin and coat (iii) comprises ketamine. In another embodiment, coat (i) comprises ketamine and coat (iii) comprises aspirin. In yet other embodiments, there may be multiple coatings of ketamine-comprising coats, and multiple coating of aspirin-comprising coats in any order. In some embodiments, a seal coat (ii) is present between any coat comprising aspirin and any coat comprising ketamine.

In some embodiments, a coat comprising aspirin is substantially free of water.

In some embodiments, a coat comprising aspirin further comprises a binder, a plasticizer, a disintegrant, and a pH modifier. In some embodiments, an aspirin coat comprises about 25% w/w to about 55% w/w aspirin. In some embodiments, an aspirin coat comprises about 5% w/w to about 15% w/w of a binder (e.g., hydroxypropyl cellulose, hydroxypropyl methylcellulose, methyl cellulose, or a combination thereof). In some embodiments, an aspirin coat comprises about 15% w/w to about 30% w/w of a disintegrant (e.g., microcrystalline cellulose). In some embodiments, an aspirin coat comprises about 10% w/w to about 25% w/w of a plasticizer (e.g., dibutyl sebacate, tributyl citrate). In some embodiments, an aspirin coat comprises about 15% w/w to about 30% w/w of a pH modifier (e.g., citric acid). In some embodiments, an aspirin coat comprises about 0.1% w/w to about 0.5% w/w sweetener (e.g., aspartame, sucralose). In some embodiments, an aspirin coat comprises about 0.1% w/w to about 1.5% w/w flavoring agent (e.g., grape flavoring, mixed berry flavoring). In some embodiments, an aspirin coat comprises about 0.1% w/w to about 0.8% w/w pigment. In such embodiments, the % w/w is based on the weight of the coat.

In some embodiments, the binder is hydroxypropyl cellulose (HPC), hydroxypropyl methylcellulose (HPMC), methyl cellulose, or polyvinylpyrrolidone (PVP), or a combination thereof.

In some embodiments, the plasticizer in an aspirin coat is dibutyl sebacate (DBS), dimethyl sebacate (DMS), dioctyl sebacate (DOS), triacetin, acetyl tributyl citrate, tributyl citrate, triethyl citrate, polyethylene glycol, or any combination thereof. In some embodiments, the plasticizer is DBS. In some embodiments, the plasticizer is tribuyl citrate. In some embodiments, the plasticizer is polyethylene glycol and DBS. In some embodiments, the plasticizer is polyethylene glycol and triacetin.

In some embodiments, the disintegrant in an aspirin coat is microcrystalline cellulose (MCC). Other disintegrants include and are not limited to starch, pre-gelatinized starch, sodium starch glycolate, and croscarmellose sodium.

In some embodiments, the pH modifier in an aspirin coat is citric acid or a salt thereof.

In some embodiments, a coat comprising aspirin is prepared by spray coating a substrate with a solution comprising aspirin, a binder, a plasticizer, a disintegrant, a pH modifier, and an alcohol. In some embodiments, an aspirin coat is sprayed on a substrate which is the core composition. In some embodiments, an aspirin coat is sprayed on a substrate which is a core composition having a ketamine coat and a seal coat.

In some embodiments, the alcohol use in the preparation of a spray solution comprising aspirin is isopropyl alcohol (IPA), or ethanol.

In some embodiments, a coat comprising ketamine is substantially free of an alcohol.

In some embodiments, a coat comprising ketamine further comprises a binder, a plasticizer, and a disintegrant. In some embodiments, a coat comprising ketamine comprises about 25% w/w about 45% w/w ketamine,. In some embodiments, a coat comprising ketamine comprises about 20% w/w about 40% w/w of a binder (e.g., hydroxypropyl methylcellulose, methyl cellulose, hydroxypropyl cellulose, or any combination thereof). In some embodiments, a coat comprising ketamine optionally comprises from about 1% w/w to about 5% w/w of polyethylene glycol or triacetin. In some embodiments, a coat comprising ketamine comprises about 25% w/w to about 45% w/w of a disintegrant (e.g., microcrystalline cellulose). In some embodiments, a coat comprising ketamine comprises about 0.05% w/w to about 0.5% w/w sweetener (e.g., sucralose, aspartame). In some embodiments, a coat comprising ketamine comprises about 0.1% w/w to about 1% w/w flavoring agent (e.g., grape flavoring, mixed berry flavoring), In some embodiments, a coat comprising ketamine comprises about 0.1% w/w to about 0.5% w/w pigment. In such embodiments, the % w/w is based on the weight of the coat;

In some embodiments, the binder comprises hydroxypropyl methylcellulose (HPMC), ethyl cellulose, methyl cellulose, hydroxypropyl cellulose, polyethylene glycol, triacetin, triethyl citrate, or any combination thereof.

In some embodiments, the binder comprises hydroxypropyl methylcellulose.

In some embodiments, the binder comprises methyl cellulose, and hydroxypropyl cellulose.

In some embodiments, the plasticizer is dibutyl sebacate (DBS), dimethyl sebacate (DMS), dioctyl sebacate (DOS), triethyl citrate, triacetin, acetyl tributyl citrate, tributyl citrate, triethyl citrate, polyethylene glycol, or any combination thereof.

In some embodiments, the binder is microcrystalline cellulose (MCC). Other disintegrants include and are not limited to starch, pre-gelatinized starch, sodium starch glycolate, and croscarmellose sodium.

In some embodiments, a coat comprising ketamine is prepared by spray coating a substrate with an aqueous solution comprising ketamine, a binder, a plasticizer, and a disintegrant. In some embodiments, a ketamine coat is sprayed on a substrate which is the core composition. In some embodiments, a ketamine coat is sprayed on a substrate which is a core composition having an aspirin coat and a seal coat.

In some embodiments, a seal coat comprises a binder, and optionally a plasticizer. In some embodiments, a seal coat comprises about 80% w/w to about 95% w/w of a binder (e.g., hydroxypropyl methyl cellulose, hydroxypropyl cellulose, methyl cellulose, or a combination thereof). In some embodiments, a seal coat comprises about 5% w/w to about 15% w/w polyethylene glycol or triacetin. In some embodiments, a seal coat comprises about 0.1% w/w to about 0.5% w/w of a sweetener (e.g., aspartame, sucralose). In some embodiments, a seal coat comprises about 0.1% w/w to about 1% w/w flavoring agent (e.g., grape flavoring, mixed berry flavoring). In some embodiments, a seal coat comprises and about 0.1% w/w to about 0.5% w/w pigment. In such embodiments, the % w/w is based on the weight of the coat.

In some embodiments, the binder in a seal coat is hydroxypropyl methylcellulose, methyl cellulose, hydroxypropyl cellulose, or any combination thereof.

In some embodiments, the optional plasticizer in a seal coat is triacetin, triethyl citrate, acetyl tributyl citrate, tributyl citrate, polyethylene glycol, or any combination thereof. In some embodiments, the seal coat comprised polyethylene glycol and triethyl citrate.

In some embodiments, the composition optionally further comprises sweeteners, pigments, and flavoring agents in the core composition, the first coat, the second coat, and the third coat.

In some embodiments, up to 50% of the total ketamine in the composition is released intraorally, and up to 50% of the total aspirin in the composition is released intraorally.

Provided is a dual release pharmaceutical composition comprising ketamine and aspirin, wherein, upon oral administration to a subject, a first portion disintegrates or dissolves intraorally providing rapid release of aspirin and ketamine, and a second portion is substantially more difficult than the first portion to disintegrate or dissolve intraorally but is ingestible and releases aspirin and ketamine in the gastrointestinal tract of the subject,
wherein the composition comprises:
(i) a core composition comprising:
   (i-a) about 3% w/w to about 25% w/w ketamine;
   (i-b) about 30% w/w to about 40% w/w aspirin;
   (i-c) about 20% w/w to about 40% w/w microcrystalline cellulose;
   (i-d) about 10% w/w to about 20% w/w of pregelatinized starch;
   (i-e) about 1% w/w to about 10% w/w citric acid;
   (i-f) about 1% w/w/to about 4% w/w croscarmellose sodium;
   (i-g) about 0.5% w/w to about 1.5% w/w silicon dioxide; and
   (i-h) about 0.1% w/w to about 0.5% w/w stearic acid;
   wherein the % w/w is based on the total weight of the core composition;
(ii) a first coat comprising ketamine and enclosing the core composition, and providing about 10% solid weight gain to the core composition (i), wherein the first coat comprises
   (ii-a) about 1% w/w about 5% w/w ketamine;
   (ii-b) about 1% w/w about 5% w/w hydroxypropyl methylcellulose;
   (ii-c) about 0.1% w/w to about 0.5% w/w polyethylene glycol;
   (ii-d) about 1% w/w to about 5% w/w microcrystalline cellulose;
   (ii-e) about 0.01% w/w to about 0.05% w/w sweetener;
   (ii-f) about 0.01% w/w to about 0.1% w/w flavoring agent; and
   (ii-g) about 0.01% w/w to about 0.05% w/w pigment;
   wherein the % w/w is based on the weight of the coat;
(iii) a second coat comprising a seal coat and enclosing the core composition coated with the first coat, and providing about 10% solid weight gain to the coated tablet (ii), wherein the second coat comprises
   (iii-a) about 8% w/w to about 9.5% w/w hydroxypropyl methyl cellulose, or hydroxypropyl cellulose, or a combination thereof;
   (iii-b) about 0.5% w/w to about 1% w/w polyethylene glycol;
   (iii-c) about 0.01% w/w to about 0.05% w/w sweetener;
   (iii-d) about 0.01% w/w to about 0.1% w/w flavoring agent; and
   (iii-e) about 0.01% w/w to about 0.05% w/w pigment;
   wherein the % w/w is based on the weight of the coat;
(iv) a third coat comprising aspirin and enclosing the core composition coated with the first coat and the second coat, and providing about 15% solid weight gain to the coated tablet (iii), wherein the third coat comprises
   (iv-a) about 3% w/w to about 6% w/w aspirin;
   (iv-b) about 0.5% w/w to about 2% w/w hydroxypropyl cellulose;
   (iv-c) about 2% w/w to about 6% w/w microcrystalline cellulose;
   (iv-d) about 1.5% w/w to about 4% w/w of dibutyl sebacate;
   (iv-e) about 2% w/w to about 5% w/w citric acid;
   (iv-f) about 0.01% w/w to about 0.05% w/w sweetener;
   (iv-g) about 0.01% w/w to about 0.2% w/w flavoring agent; and
   (iv-h) about 0.01% w/w to about 0.1% w/w pigment;
wherein the % w/w is based on the weight of the coat.

In some embodiments, the composition comprises:
(i) a core composition comprising:
   (i-a) about 3% w/w to about 35% w/w ketamine;
   (i-b) about 30% w/w to about 50% w/w aspirin;
   (i-c) about 15% w/w to about 60% w/w microcrystalline cellulose;
   (i-d) about 5% w/w to about 20% w/w of pregelatinized starch;
   (i-e) about 0.5% w/w to about 5% w/w citric acid;
   (i-f) about 0.2% w/w/to about 4% w/w croscarmellose sodium;
   (i-g) about 0.1% w/w to about 1.5% w/w silicon dioxide; and
   (i-h) about 0.01% w/w to about 0.5% w/w stearic acid;
   wherein the % w/w is based on the total weight of the core composition;
(ii) a first coat comprising ketamine and enclosing the core composition, and providing about 10-20% weight gain to the core composition (i), wherein the first coat comprises
   (ii-a) about 25% w/w about 45% w/w ketamine;
   (ii-b) about 20% w/w about 40% w/w hydroxypropyl methylcellulose;
   (ii-c) about 1% w/w to about 5% w/w polyethylene glycol;
   (ii-d) about 25% w/w to about 45% w/w microcrystalline cellulose;
   (ii-e) about 0.05% w/w to about 0.5% w/w sweetener;
   (ii-f) about 0.1% w/w to about 1% w/w flavoring agent; and
   (ii-g) about 0.1% w/w to about 0.5% w/w pigment;
   wherein the % w/w is based on the weight of the coat;
(iii) a second coat comprising a seal coat and enclosing the core composition coated with the first coat, and providing about 1-5% weight gain to the coated tablet (ii), wherein the second coat comprises
   (iii-a) about 80% w/w to about 95% w/w hydroxypropyl methyl cellulose or hydroxypropyl cellulose, or a combination thereof;
   (iii-b) about 5% w/w to about 15% w/w polyethylene glycol;
   (iii-c) about 0.1% w/w to about 0.5% w/w sweetener;
   (iii-d) about 0.1% w/w to about 1% w/w flavoring agent; and
   (iii-e) about 0.1% w/w to about 0.5% w/w pigment;
   wherein the % w/w is based on the weight of the coat;
(iv) a third coat comprising aspirin and enclosing the core composition coated with the first coat and the second coat, and providing about 80-120% weight gain to the coated tablet (iii), wherein the third coat comprises
   (iv-a) about 25% w/w to about 55% w/w aspirin;
   (iv-b) about 5% w/w to about 15% w/w hydroxypropyl cellulose;
   (iv-c) about 15% w/w to about 30% w/w microcrystalline cellulose;
   (iv-d) about 10% w/w to about 25% w/w of dibutyl sebacate;
   (iv-e) about 15% w/w to about 30% w/w citric acid;
   (iv-f) about 0.1% w/w to about 0.5% w/w sweetener;
   (iv-g) about 0.1% w/w to about 1.5% w/w flavoring agent; and
   (iv-h) about 0.1% w/w to about 0.8% w/w pigment;
wherein the % w/w is based on the weight of the coat.

In such embodiments, the coats (ii)-(iv) form the first portion which disintegrates or dissolves intraorally providing rapid release of ketamine and aspirin. In such embodiments, the core composition (i) forms the second portion that is substantially more difficult than the first portion to disintegrate or dissolve intraorally but is ingestible and releases ketamine and aspirin in the gastrointestinal tract.

In some embodiments, a dual release ketamine and aspirin pharmaceutical composition comprises ketamine and aspirin in a core composition, formulated for GI release, and coatings on the core composition comprising ketamine or aspirin wherein the coatings on the core composition allow for intraoral release of ketamine and aspirin. One or more coats comprising ketamine and/or aspirin may be present on the core composition. In some embodiments, a seal coat may be present as the final coat. In some embodiments, a seal coat is present between the core composition and the coats comprising ketamine and/or that is formulated for intraoral release. In some embodiments, a seal coat is present between the coats comprising ketamine and the coats comprising aspirin. In some embodiments, a seal coat is present between the coats comprising ketamine and the coats comprising aspirin, and a seal coat is also present as a final coat.

The pharmaceutical composition of the above embodiments can further include a third portion that comprises an effective amount of ketamine and/or aspirin. In one embodiment, the third portion is in the form of controlled release. In another embodiment, the third portion further comprises enteric coating. In yet another embodiment, the third portion is enclosed in the first portion and/or the second portion.

In some embodiments, the NMDA receptor antagonist (e.g., ketamine or a metabolite or analog thereof) is at an amount lower than the regular dose of the NMDA receptor antagonist (e.g., 100-400 mg), such as but not limited to, from about 20 mg to about 300 mg. In one embodiment, the amount of the NMDA receptor antagonist of the first portion is at least about 20 mg, or least about 30 mg, 40 mg, 50 mg, or 100 mg. In another embodiment, the amount of the NMDA receptor antagonist of the first portion is no more than about 50 mg, 75 mg, 100 mg, 125 mg, 150 mg, 175 mg, 200 mg, 225 mg, 250 mg, 275 mg or 300 mg.

In some embodiments, the composition includes two portions of aspirin, a first portion containing a first amount of the aspirin, and a second portion containing a second amount of the aspirin. Upon oral administration to a subject, the first portion disintegrates or dissolves intraorally providing rapid release of the aspirin of the first portion, and the second portion is substantially more difficult than the first portion to disintegrate or dissolve intraorally but is ingestible and releasable in the gastrointestinal tract of the subject.

### 6. Additional Embodiments for Compositions

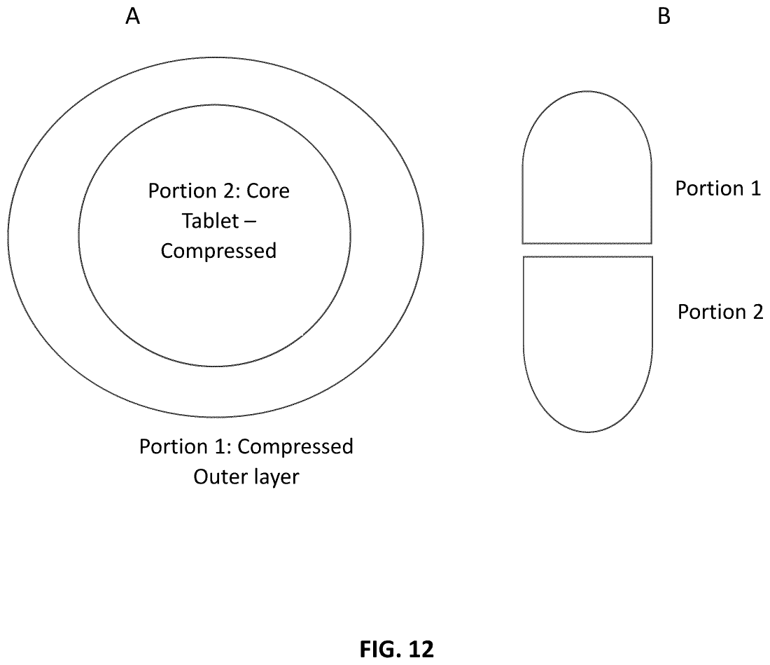

Additional pharmaceutical compositions are contemplated within the scope of the present disclosure. FIG. 12A shows an embodiment wherein the pharmaceutical composition is a tablet in tablet compressed dosage form. In such embodiments, a core composition is a compressed core tablet which forms the second portion that is substantially more difficult than the first portion to disintegrate or dissolve intraorally but is ingestible and releases ketamine and/or aspirin in the gastrointestinal tract. In such embodiments, an outer layer is compressed around the core tablet and forms the first portion which disintegrates or dissolves intraorally providing rapid release of ketamine and/or aspirin.

FIG. 12B shows an embodiment where two adjacent regions are present within the dosage form. In such embodiments, a first composition is a compressed solid form comprising a modified release ingredient so that it will not burst in the mouth and can withstand 30 seconds-5 minutes, or 30 seconds to 1 minute, of residence time in the oral cavity, and the active ingredient can be absorbed in the GI tract. The first composition forms the second portion that is substantially more difficult than the first portion to disintegrate or dissolve intraorally but is ingestible and releases ketamine and/or aspirin in the gastrointestinal tract. In such embodiments, a second composition is placed adjacent to, or in proximity to, the core tablet and is a compressed solid form utilizing a super disintegrant so that it can burst, and active agent can be released in the mouth. The second composition thus forms the first portion which disintegrates or dissolves intraorally providing rapid release of ketamine and/or aspirin.

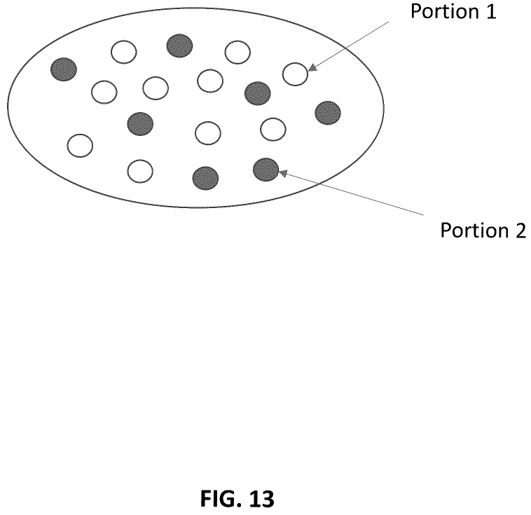

FIG. 13 shows an embodiment where beads or granules are present within the dosage form. In such embodiments, a first set of beads or granules comprises a modified release ingredient so that the granules or beads will not burst in the mouth and can withstand 30 seconds-5 minutes, or 30 seconds to 1 minute, of residence time in the oral cavity, and the active ingredient can be absorbed in the GI tract. The first set of beads or granules forms the second portion that is substantially more difficult than the first portion to disintegrate or dissolve intraorally but is ingestible and releases ketamine and/or aspirin in the gastrointestinal tract. In such embodiments, a second set of beads or granules is placed adjacent to, or in proximity to, the first set of beads or granules, or in interspersed with the first set of beads and granules, and comprises a super disintegrant so that it can burst, and active agent can be released in the mouth. The second set of beads or granules thus forms the first portion which disintegrates or dissolves intraorally providing rapid release of ketamine and/or aspirin. The two sets of beads or granules are compressed into a tablet or filled in a capsule. The tablet or capsule shell comprises a super disintegrant and bursts in the mouth. Granules or beads forming the first portion would dissolve in the mouth while granules or beads forming the second portion would withstand dissolution in the oral cavity and dissolve in the GI tract.

### 7. Additional Additives to the Compositions

In yet another embodiment, either or both of the first portion and second portion further comprises excipients, lubricants, pH adjusters, taste-masking agents, sweeteners, acidifiers, refrigerants, foaming agents, preservatives, fluidizers, antioxidants, colorants, stabilizers, surfactants, buffering agents, flavors, binders or drug solubilizers. A person skilled in the art may immediately list specific examples of these additives.

Any excipient used for pharmaceutical preparations can be used without limitation, but examples of excipients used in the tablet of the present invention can include sugars such as crythritol, mannitol, xylitol, sorbitol, lactitol, paratinit, paratinose, maltitol, maltose, trehalose, lactose, sucrose, glucose, olygosaccharides, fructose and maltose and the like. One or two or more kinds of these excipients can be used.

Various embodiments of the composition may include pharmaceutically acceptable binders (adhesives). Binders are agents that impart cohesive properties to powdered materials through particle-particle bonding. Examples of suitable binders include celluloses and crosslinked polyvinyl pyrrolidone, matrix binders (dry starch, dry sugars), film binders (polyvinyl pyrrolidone (PVP), starch paste, celluloses, bentonite, sucrose), and chemical binders (polymeric cellulose derivatives, such as carboxy methyl cellulose, hydroxypropylcellulose (HPC) and hydroxypropylmethylcellulose (HPMC); sugar syrups; corn syrup; water soluble polysaccharides such as acacia, tragacanth, guar and alginates; gelatin; gelatin hydrolysate; agar; sucrose; dextrose; and non-cellulosic binders, such as polyvinyl pyrrolidone, polyethylene glycol (PEG), vinyl pyrrolidone copolymers, pregelatinized starch, sorbitol, glucose, microcrystalline cellulose, such as FMC BioPolymer's Avicel® PH101 and Avicel® PH102, and silicified microcrystalline cellulose, such as Penwest Pharmaceutical's ProSolv SMCC™). In specific embodiments, a binder is selected from the group consisting of corn starch, potato starch, polyvinyl pyrrolidone, hydroxypropylmethyl cellulose, and hydroxylpropyl cellulose. A binder may be included in any portion of the dosage form, such as the intragranular portion and/or extragranular portion of either or both of the first and second layers.

In some embodiments, the composition further comprises a pharmaceutically acceptable diluent or filler. Pharmaceutically acceptable diluents include, but are not limited to, lactose (such as lactose monohydrate, lactose anhydrous, and DMV International's Pharmatose® DCL21 crystalline alpha monohydrate milled lactose), mannitol, talc, magnesium stearate, sodium chloride, potassium chloride, citric acid, spray-dried lactose, starch, hydrolyzed starches, directly compressible starch, microcrystalline cellulose (such as Avicel® PH101 and Avicel® PH102), cellulosics, sorbitol, sucrose, glucose, sucrose-based materials, saccharides, calcium sulfate, dibasic calcium phosphate (such as Emcompress®) and dextrose, and/or mixtures of any of the foregoing. In specific embodiments, a diluent is selected from the group consisting of microcrystalline cellulose, lactose, mannitol, dicalcium phosphate, dextrose, compressible sugar, and spray-dried lactose with microcrystalline cellulose. A diluent may be included in any portion of the dosage form, such as the intragranular portion and/or extragranular portion of either or both of the first and second layers.

In some embodiments, the composition comprises magnesium stearate. In specific embodiments, the magnesium stearate is present in a range of about 0.5% to 2% w/w, based on the total weight of the layer.

In some embodiments, the diluent is microcrystalline cellulose or microlac (spray-dried lactose with microcrystalline cellulose). In specific embodiments, the microcrystalline cellulose or microlac is present in a range of about 20% to 95% w/w, based on the total weight of the layer.

Various embodiments of the invention may include pharmaceutically acceptable anti-adherents (anti-sticking agents, glidants, flow promoters, lubricants) such as talc, colloidal silicon dioxide, such as Aerosil® 200, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, calcium stearate, silica gel, starch, hydrogenated vegetable oils, sodium benzoate, sodium acetate, leucine, PEG-4000, and magnesium lauryl sulfate. In specific embodiments, an anti-adherents is selected from glidants and lubricants. Suitable glidants include, but are not limited to, colloidal silicon dioxide (Aerosil®), magnesium trisilicate, talc, and tribasic calcium phosphate. Suitable lubricants include, but are not limited to magnesium, aluminum, calcium, zinc stearate, and talc. An anti-adherent may be included in any portion of the dosage form, such as the intragranular portion and/or extragranular portion of either or both of the first and second layers. In specific embodiments, an anti-adherent is included in the extragranular portion of the first layer and/or the extragranular portion of the second layer.

In some embodiments, the glidant is talc. In specific embodiments, talc is present in a range of about 1% to 7% w/w, based on the total weight of each layer.

### D. Therapeutic Methods

Therapeutic methods are also provided. In one embodiment, provided is a method of administering ketamine to a subject with improved efficacy or bioavailability, comprising administering to the subject a dual release ketamine composition wherein a first portion of the composition disintegrates or dissolves intraorally providing a first release of ketamine which is intraoral, and the second portion of the composition is ingested and provides a second release of the ketamine in the gastrointestinal tract of the subject.

In one embodiment, the ketamine released in the first portion (e.g., intraorally) is at an amount lower than the regular dose of ketamine (e.g., 10-400 mg), such as but not limited to, from about 10 mg to about 300 mg. In one embodiment, the amount of ketamine released in the first portion (e.g., intraorally) is at least about 10 mg, or least about 20 mg, 30 mg, 40 mg, 50 mg, or 100 mg. In another embodiment, the amount of ketamine released in the first portion (e.g., intraorally) is no more than about 50 mg, 75 mg, 100 mg, 125 mg, 150 mg, 175 mg, 200 mg, 225 mg, 250 mg, 275 mg or 300 mg. In one embodiment, the ketamine in the second portion (e.g., in the GI tract) is at an amount lower than the regular dose of ketamine (e.g., 100-400 mg), such as but not limited to, from about 10 mg to about 300 mg. In one embodiment, the amount of ketamine released in a second portion (e.g., in the GI tract) is at least about 10 mg, or least about 20 mg, 30 mg, 40 mg, 50 mg, or 100 mg. In another embodiment, the amount of ketamine released in a second portion (e.g., in the GI tract) is no more than about 50 mg, 75 mg, 100 mg, 125 mg, 150 mg, 175 mg, 200 mg, 225 mg, 250 mg, 275 mg or 300 mg.

In one embodiment, the ketamine of the first release is at least about 10%, or 20%, or 30%, or 40%, of 50%, or 60%, or 70%, or 80%, or 90% of a therapeutically effective amount. In one embodiment, the ketamine of the first release is at most about 10%, or 20%, or 30%, or 40%, of 50%, or 60%, or 70%, or 80%, or 90% of a therapeutically effective amount. In one embodiment, the ketamine of the second release is at least about 10%, or 20%, or 30%, or 40%, of 50%, or 60%, or 70%, or 80%, or 90% of a therapeutically effective amount. In one embodiment, the ketamine of the second release is at most about 10%, or 20%, or 30%, or 40%, of 50%, or 60%, or 70%, or 80%, or 90% of a therapeutically effective amount.

In one embodiment, the first release of ketamine constitutes at least about 10% of the total ketamine administered. Alternatively, the first release of ketamine constitutes at least about 20%, or 30%, or 40%, or 50%, or 60%, or 70%, or 80%, or 90% of the total ketamine. In some embodiments, however, the first release of ketamine can be less than about 20%, or 30%, or 40%, or 50%, or 60%, or 70%, or 80%, or 90% of the total ketamine. In a particular embodiment, the first release constitutes from about 40% to about 60%, or alternatively from about 45% to about 55% of the total ketamine.

In one embodiment, the total amount of ketamine administered is less than about 50 mg, or 60 mg, or 70 mg, or 80 mg, or 90 mg, or 100 mg, or 120 mg, or 140 mg, or 150 mg, or 160 mg, or 165 mg, or 170 mg, or 180 mg, or 190 mg, 200 mg, 210 mg, 220 mg, 230 mg, 240 mg, 250 mg, 260 mg, 270 mg, 280 mg, 290 mg, or 300 mg. In another embodiment, the total amount of ketamine administered is greater than about 10 mg, 20 mg, or 30 mg, or 40 g, or 50 mg, or 60 mg, or 70 mg, or 80 mg, or 90 mg, or 100 mg, or 110 mg, or 120 mg, or 130 mg, or 140 mg, or 150 mg.

Also provided, in one embodiment, is a method for treating acute pain in a patient, comprising orally administering an effective amount of ketamine to the patient.

The effective amount may be at least 0.1 mg/kg, or at least 0.2 mg/kg, 0.3, 0.35, 0.4, 0.45, 0.5, 0.55, 0.6, 0.65, 0.7, 0.75, 0.8, 0.85, 0.9, 0.95 or 1 mg/kg. In some embodiments, the effective amount is not greater than 2 mg/kg, or not greater than 1.9 mg/kg, 1.85, 1.8, 1.75, 1.7, 1.65, 1.6, 1.55, 1.5, 1.45, 1.4, 1.35, 1.3, 1.25, 1.2, 1.15, 1.1, 1.05, 1, 0.95, 0.9, 0.85, 0.8, 0.75, 0.7, 0.65, 0.6, 0.55, or 0.5 mg/kg. In some embodiments, the effective amount is from 0.3 mg/kg to 0.8 mg/kg, preferably from 0.4 mg/kg to 0.6 mg/kg, and more preferably 0.5 mg/kg.

Also provided, in one embodiment, is a method for treating acute or chronic headache in a patient, comprising orally administering an effective amount of ketamine to the patient.

The effective amount may be at least 0.1 mg/kg, or at least 0.2 mg/kg, 0.3, 0.35, 0.4, 0.45, 0.5, 0.55, 0.6, 0.65, 0.7, 0.75, 0.8, 0.85, 0.9, 0.95 or 1 mg/kg. In some embodiments, the effective amount is not greater than 2 mg/kg, or not greater than 1.9 mg/kg, 1.85, 1.8, 1.75, 1.7, 1.65, 1.6, 1.55, 1.5, 1.45, 1.4, 1.35, 1.3, 1.25, 1.2, 1.15, 1.1, 1.05, 1, 0.95, 0.9, 0.85, 0.8, 0.75, 0.7, 0.65, 0.6, 0.55, or 0.5 mg/kg. In some embodiments, the effective amount is from 0.5 mg/kg to 1.5 mg/kg, preferably from 0.7 mg/kg to 1 mg/kg, and more preferably 0.85 mg/kg.

Also provided, in one embodiment, is a method for treating depression in a patient, comprising orally administering an effective amount of ketamine to the patient.

The effective amount may be at least 0.1 mg/kg, or at least 0.2 mg/kg, 0.3, 0.35, 0.4, 0.45, 0.5, 0.55, 0.6, 0.65, 0.7, 0.75, 0.8, 0.85, 0.9, 0.95 or 1 mg/kg. In some embodiments, the effective amount is not greater than 2 mg/kg, or not greater than 1.9 mg/kg, 1.85, 1.8, 1.75, 1.7, 1.65, 1.6, 1.55, 1.5, 1.45, 1.4, 1.35, 1.3, 1.25, 1.2, 1.15, 1.1, 1.05, 1, 0.95, 0.9, 0.85, 0.8, 0.75, 0.7, 0.65, 0.6, 0.55, or 0.5 mg/kg. In some embodiments, the effective amount is from 0.75 mg/kg to 1.75 mg/kg, preferably from 1 mg/kg to 1.5 mg/kg, and more preferably 1.2 mg/kg.

Also provided, in one embodiment, is a method for treating cannabinoid hyperemesis syndrome exacerbations in a patient, comprising orally administering an effective amount of ketamine to the patient.

The effective amount may be at least 0.1 mg/kg, or at least 0.2 mg/kg, 0.3, 0.35, 0.4, 0.45, 0.5, 0.55, 0.6, 0.65, 0.7, 0.75, 0.8, 0.85, 0.9, 0.95 or 1 mg/kg. In some embodiments, the effective amount is not greater than 2 mg/kg, or not greater than 1.9 mg/kg, 1.85, 1.8, 1.75, 1.7, 1.65, 1.6, 1.55, 1.5, 1.45, 1.4, 1.35, 1.3, 1.25, 1.2, 1.15, 1.1, 1.05, 1, 0.95, 0.9, 0.85, 0.8, 0.75, 0.7, 0.65, 0.6, 0.55, or 0.5 mg/kg. In some embodiments, the effective amount is from 0.2 mg/kg to 1 mg/kg, preferably from 0.2 mg/kg to 0.6 mg/kg, and more preferably 0.4 mg/kg.

In some embodiments, the addition of an GRIN2A modulator, e.g., aspirin, can further enhance the bioavailability and/or efficacy of the ketamine. In accordance with one embodiment of the disclosure, provided is a method of administering ketamine to a subject with improved efficacy or bioavailability, or reduced side effects, comprising administering to the subject ketamine and a glutamate ionotropic receptor NMDA type subunit 2A (GRIN2A) modulator. In some embodiments, the GRIN2A modulator is a GRIN2A activator. Non-limiting examples include aspirin, nicotine, propofol, melatonin and GQ1b.

In some embodiments, the GRIN2A modulator is absorbed intraorally. In some embodiments, the GRIN2A modulator is absorbed in the GI tract. In some embodiments, the GRIN2A modulator is partially absorbed intraorally and partially absorbed in the GI tract.

In some embodiments, the GRIN2A modulator is aspirin. In one embodiment, aspirin is administered in a manner that disintegrates or dissolves intraorally providing a first rapid release of the aspirin in a first portion. In some embodiments, the aspirin so administered is at least about 40 mg. In some embodiments, the aspirin is at least about 80 mg, 160 mg, 240 mg or 320 mg. In such embodiments, aspirin is administered such that a second portion is ingestible and provides a second release in the gastrointestinal tract of the subject. In some embodiments, the aspirin so administered is at least about 40 mg. In some embodiments, the aspirin so administered is at least about 80 mg, 160 mg, 240 mg or 320 mg.

In one embodiment, the aspirin released in the first portion (e.g., intraorally) is at an amount lower than the regular dose of aspirin (e.g., 10-400 mg), such as but not limited to, from about 10 mg to about 300 mg. In one embodiment, the amount of aspirin released in the first portion (e.g., intraorally) is at least about 10 mg, or least about 20 mg, 30 mg, 40 mg, 50 mg, or 100 mg. In another embodiment, the amount of aspirin released in the first portion (e.g., intraorally) is no more than about 50 mg, 75 mg, 100 mg, 125 mg, 150 mg, 175 mg, 200 mg, 225 mg, 250 mg, 275 mg or 300 mg. In one embodiment, the aspirin in the second portion (e.g., in the GI tract) is at an amount lower than the regular dose of aspirin (e.g., 100-400 mg), such as but not limited to, from about 10 mg to about 300 mg. In one embodiment, the amount of aspirin released in a second portion (e.g., in the GI tract) is at least about 10 mg, or least about 20 mg, 30 mg, 40 mg, 50 mg, or 100 mg. In another embodiment, the amount of aspirin released in a second portion (e.g., in the GI tract) is no more than about 50 mg, 75 mg, 100 mg, 125 mg, 150 mg, 175 mg, 200 mg, 225 mg, 250 mg, 275 mg or 300 mg.

In one embodiment, the aspirin of the first release is at least about 10%, or 20%, or 30%, or 40%, of 50%, or 60%, or 70%, or 80%, or 90% of a therapeutically effective amount. In one embodiment, the aspirin of the first release is at most about 10%, or 20%, or 30%, or 40%, of 50%, or 60%, or 70%, or 80%, or 90% of a therapeutically effective amount. In one embodiment, the aspirin of the second release is at least about 10%, or 20%, or 30%, or 40%, of 50%, or 60%, or 70%, or 80%, or 90% of a therapeutically effective amount. In one embodiment, the aspirin of the second release is at most about 10%, or 20%, or 30%, or 40%, of 50%, or 60%, or 70%, or 80%, or 90% of a therapeutically effective amount.

In one embodiment, the first release of aspirin constitutes at least about 10% of the total aspirin administered. Alternatively, the first release of aspirin constitutes at least about 20%, or 30%, or 40%, or 50%, or 60%, or 70%, or 80%, or 90% of the total aspirin. In some embodiments, however, the first release of aspirin can be less than about 20%, or 30%, or 40%, or 50%, or 60%, or 70%, or 80%, or 90% of the total aspirin. In a particular embodiment, the first release constitutes from about 40% to about 60%, or alternatively from about 45% to about 55% of the total aspirin.

In one embodiment, the total amount of aspirin administered is less than about 50 mg, or 60 mg, or 70 mg, or 80 mg, or 90 mg, or 100 mg, or 120 mg, or 140 mg, or 150 mg, or 160 mg, or 165 mg, or 170 mg, or 180 mg, or 190 mg, 200 mg, 210 mg, 220 mg, 230 mg, 240 mg, 250 mg, 260 mg, 270 mg, 280 mg, 290 mg, or 300 mg. In another embodiment, the total amount of aspirin administered is greater than about 10 mg, 20 mg, or 30 mg, or 40 g, or 50 mg, or 60 mg, or 70 mg, or 80 mg, or 90 mg, or 100 mg, or 110 mg, or 120 mg, or 130 mg, or 140 mg, or 150 mg.

In some embodiments, the effective amount of aspirin administered is from about 25 mg to about 800 mg per day, about 25 mg to about 700 mg per day, about 25 mg to about 600 mg per day, about 25 mg to about 500 mg per day, about 25 mg to about 500 mg per day, about 25 mg to about 400 mg per day, about 25 mg to about 300 mg per day, about 25 mg to about 200 mg per day, or about 25 mg to about 100 mg per day. In some embodiments, the effective amount of aspirin administered is about 800 mg per day, about 750 mg/day, about 700 mg per day, about 650 mg per day, about 600 mg per day, about 550 mg per day, about 500 mg per day, about 450 mg per day, about 400 mg per day, about 350 mg per day, about 300 mg per day, about 250 mg per day, about 200 mg per day, about 150 mg per day, about 100 mg per day, or about 50 mg/day. In some embodiments, the effective amount of aspirin administered is about 650 mg/day.

In some embodiments, the amount of aspirin in an individual capsule or tablet is about 25-500 mg per capsule or tablet, about 25-400 mg per capsule or tablet, about 25-300 mg per capsule or tablet, about 25-200 mg per capsule or tablet, about 25-100 mg per capsule or tablet, about 25-50 mg per capsule or tablet, about 35-500 mg per capsule or tablet, about 35-400 mg per capsule or tablet, about 50-400 mg per capsule or tablet, or about 75-400 mg per capsule or tablet. In some embodiments, the amount of aspirin in an individual capsule or tablet is about 162 mg.

In some embodiments of the methods described herein, a combination dosage form comprising ketamine and aspirin is administered to a subject, where both ketamine and aspirin have dual release profiles, i.e., a first portion is release intraorally and a second portion is released in the GI tract. In such embodiments, the effective amounts of ketamine and/or aspirin are as described above.

In some other embodiments of the methods described herein, a dosage form comprising ketamine alone, where the ketamine has a dual release profile, i.e., a first portion is release intraorally and a second portion is released in the GI tract, is administered to a subject in combination with a dosage form comprising aspirin alone, where the aspirin has a dual release profile, i.e., a first portion is released intraorally and a second portion is released in the GI tract.

In some embodiments of the methods described herein, a dosage form comprising ketamine alone is administered to a subject, where the ketamine has a dual release profile, i.e., a first portion is release intraorally and a second portion is released in the GI tract.

In some embodiments of the methods described herein, a dosage form comprising aspirin alone is administered to a subject, where the aspirin has a dual release profile, i.e., a first portion is release intraorally and a second portion is released in the GI tract.

Thus, also provided is a method of administering ketamine to a subject in need of treating a disease or condition, comprising administering to the subject a therapeutically effective amount of ketamine, wherein a portion of the ketamine disintegrates or dissolves intraorally within 10 minutes permitting rapid release of the ketamine in the portion, and the remaining ketamine is ingested and released in the gastrointestinal tract of the subject.

The disease or condition, without limitation, can be pain, asthma, or depression. In some embodiments, the subject is need of anesthesia.

In some embodiments, the first portion of the ketamine disintegrates or dissolves intraorally within about 9 minutes, or about 8, or about 7, or about 6, or about 5, or about 4, or about 3 or about 2 minutes, or alternatively about 60 seconds, or about 50, or about 40, or about 30, or about 20, or about 10, or about 5 seconds. In any embodiment of the above methods, the method further comprises administering to the subject an effective amount of aspirin.

Also provided, in one embodiment, is a method of administering ketamine to a subject, comprising administering to the subject ketamine and aspirin.

The administration of ketamine can be useful for treating certain diseases or conditions, such as pain, asthma, or depression. In some embodiments, the subject is need of anesthesia.

### EXAMPLES OF COMPOSITIONS

### Example 1A. Dual-Route Ketamine Only Pharmaceutical composition

The core tablet is prepared by compression using the ingredients in Table 1 below. The core tablet is spray coated using an aqueous solution comprising the ingredients in Table 1 below and dried.

TABLE 1
Ketamine 10 mg - Core Tablet Compression

| | | % w/w of | % w/w | |
|---|---|---|---|---|
| S. NO | Description | core | total | mg/unit |
| 1 | Ketamine Hydrochloride*** | 5.24 | 4.55 | 11.53 |
| 2 | Microcrystalline Cellulose (Vivapur 102) | 93.13 | 80.95 | 204.92 |
| 3 | Croscarmellose Sodium (Vivasol) | 1.09 | 0.95 | 2.40 |
| 4 | Colloidal Silicon Dioxide (Aerosil 200) | 0.27 | 0.23 | 0.59 |
| 5 | Stearic acid | 0.27 | 0.23 | 0.59 |

| | | | |
|---|---|---|---|
| Total | 100 | — | 220.03 |

Ketamine 10 mg - Drug Coating

| | | % w/w of | % w/w of | % w/w | |
|---|---|---|---|---|---|
| S. NO | Description | solution | coating | total | mg/unit |
| 6 | Ketamine Core Tablets | — | — | 86.92 | 220.03 |
| 7 | Ketamine Hydrochloride*** | 3.33 | 33.30 | 4.55 | 11.53 |
| 8 | Opadry Clear YS-1-19025-A* | 3.33 | 33.30 | 4.55 | 11.53 |
| 9 | Microcrystalline Cellulose (Vivapur 105) | 3.22 | 32.20 | 3.82 | 9.67 |
| 10 | Sucralose | 0.02 | 0.20 | 0.02 | 0.06 |
| 11 | Mixed Berry Flavoring (807.1196U) | 0.07 | 0.70 | 0.09 | 0.22 |
| 12 | FD&C Blue #1 Aluminum Lake | 0.03 | 0.30 | 0.04 | 0.10 |
| 13 | Purified Water** | 90.00 | — | — | — |

| | | | | |
|---|---|---|---|---|
| Total | 100.00 | 100.00 | 100.00 | 253.14 |

| | |
|---|---|
| Solid content (%) | 10.00 |

*Opadry Clear YS-1-19025A is composed of 90.9% of Hypromellose 2910, E6 and 9.1% of PEG 400
**removed during processing
***11.53 mg of Ketamne Hydrochloride is equivalent to 10 mg of Ketamine

### Example 2A. Dual-Route Aspirin Only Pharmaceutical composition

The core tablet is prepared by compression using the ingredients in Table 2 below. The core tablet is spray coated using an aqueous solution comprising the ingredients in Table 2 below and dried.

TABLE 2
Aspirin 81 mg - Core tablet Compression

| | | % w/w | | |
|---|---|---|---|---|
| S. No | Description | of core | % w/w total | mg/unit |
| 1 | Aspirin | 36.82 | 16.58 | 81 |
| 2 | Microcrystalline Cellulose (Vivapur 102) | 61.55 | 27.71 | 135.4 |
| 3 | Croscarmellose Sodium (Vivasol) | 1.09 | 0.49 | 2.4 |
| 4 | Colloidal Silicon Dioxide (Aerosil 200) | 0.27 | 0.12 | 0.59 |
| 5 | Stearic acid | 0.27 | 0.12 | 0.59 |

| | | | |
|---|---|---|---|
| Total | 100.00 | — | 219.98 |

Aspirin 81 mg - Drug Coating

| | | % w/w | % w/w | | |
|---|---|---|---|---|---|
| | | of | of | | |
| S. No | Description | solution | coating | % w/w total | mg/unit |
| 6 | Aspirin Core Tablets | — | — | 45.02 | 219.98 |
| 7 | Aspirin | 4.52 | 30.13 | 16.58 | 81.00 |
| 8 | Hydroxypropyl Cellulose (Klucel LF) | 1.18 | 7.87 | 4.34 | 21.20 |
| 9 | Microcrystalline Cellulose (Vivapur 105) | 3.55 | 23.67 | 13.02 | 63.61 |
| 10 | Dibutyl Sebacate | 2.37 | 15.80 | 8.68 | 42.41 |
| 11 | Citric acid Anhydrous, Granular | 3.16 | 21.07 | 11.59 | 56.62 |
| 12 | Sucralose | 0.03 | 0.20 | 0.12 | 0.57 |
| 13 | Mixed Berry Flavoring (807.1196U) | 0.13 | 0.87 | 0.46 | 2.26 |
| 14 | FD&C Blue #1 Aluminum Lake | 0.06 | 0.40 | 0.20 | 0.99 |
| 15 | Isopropyl Alcohol** | 85 | — | — | — |

| | | | | |
|---|---|---|---|---|
| Total | 100.00 | 100.00 | 100.00 | 488.64 |

| | |
|---|---|
| Solid Content (%) | 15.00 |

**removed during processing

### Example 3A. Dual-Route Ketamine and Aspirin Pharmaceutical composition

The core tablet is prepared by compression using the ingredients in Table 3 below. The core tablet is spray coated using an aqueous solution comprising the ingredients in Table 3 below and dried.

TABLE 3
Ketamine Aspirin Combined Core Tablet- Compression

| | | % w/w of | % w/w | |
|---|---|---|---|---|
| S. No | Description | core | total | mg/unit |
| 1 | Ketamine Hydrochloride*** | 5.77 | 2.41 | 17.30 |
| 2 | Aspirin | 36.00 | 15.04 | 108.00 |
| 3 | Microcrystalline Cellulose (Vivapur 102) | 37.68 | 15.74 | 113.05 |
| 4 | Pregelatinized starch (Starch 1500) | 15.00 | 6.27 | 45.00 |
| 5 | Citric acid Anhydrous, Granular | 3.00 | 1.25 | 9.00 |
| 6 | Croscarmellose Sodium (Vivasol) | 1.50 | 0.63 | 4.50 |
| 7 | Colloidal Silicon Dioxide (Aerosil 200) | 0.75 | 0.31 | 2.25 |
| 8 | Stearic acid | 0.30 | 0.13 | 0.90 |

| | | | |
|---|---|---|---|
| Total | 100.00 | — | 300.00 |

Ketamine Drug Cost

| | | % w/w of | % w/w of | % w/w | |
|---|---|---|---|---|---|
| S. No | Description | solution | coating | total | mg/unit |
| 9 | Ketamine & Aspirin Core Tablets | — | — | 41.77 | 300 |
| 10 | Ketamine Hydrochloride *** | 3.33 | 33.3 | 2.41 | 17.3 |
| 11 | Opadry Clear YS-1-19025-A* | 3.33 | 33.3 | 2.41 | 17.3 |
| 12 | Microcrystalline Cellulose (Vivapur 105) | 3.22 | 32.2 | 2.02 | 14.5 |
| 13 | Sucralose | 0.02 | 0.2 | 0.01 | 0.09 |
| 14 | Mixed Berry Flavoring (807.1196U) | 0.07 | 0.7 | 0.05 | 0.33 |
| 15 | FD&C Blue #1 Aluminum Lake | 0.03 | 0.3 | 0.02 | 0.15 |
| 16 | Purified Water** | 90 | — | — | — |

| | | | | |
|---|---|---|---|---|
| Total | 100.00 | 100.00 | — | 349.67 |

| | |
|---|---|
| % solids | 10.00 |

Aqueous Seal Coat

| | | % w/w of | % w/w of | % w/w | |
|---|---|---|---|---|---|
| S. No | Description | solution | coating | total | mg/unit |
| 17 | Ketamine coated tablets | — | — | 48.68 | 349.67 |
| 18 | Hydroxypropyl Cellulose (Klucel LF) | 4.55 | 45.5 | 0.66 | 4.71 |
| 19 | HPMC E5 (Vivapharm HPMC E5) | 4.55 | 45.5 | 0.66 | 4.71 |
| 20 | Polyethylene glycol 400 | 0.82 | 8.2 | 0.12 | 0.85 |
| 21 | Sucralose | 0.02 | 0.2 | 0.003 | 0.02 |
| 22 | Mixed Berry Flavoring (807.1196U) | 0.05 | 0.5 | 0.01 | 0.05 |
| 23 | FD&C Blue #1 Aluminum Lake | 0.02 | 0.2 | 0.003 | 0.02 |
| 24 | Purified Water** | 90 | — | — | — |

| | | | | |
|---|---|---|---|---|
| Total | 100.00 | 100.00 | — | 360.03 |

| | |
|---|---|
| % solids | 10.00 |

Aspirin Drug Coat

| | | % w/w of | % w/w of | % w/w | |
|---|---|---|---|---|---|
| S. No | Description | solution | coating | total | mg/unit |
| 25 | Seal coated tablets | — | — | 50.13 | 360.03 |
| 26 | Aspirin | 4.52 | 30.13 | 15.04 | 108.00 |
| 27 | Hydroxypropyl Cellulose (Klucel LF) | 1.18 | 7.87 | 3.94 | 28.27 |
| 28 | Microcrystalline Cellulose (Vivapur 105) | 3.55 | 23.67 | 11.81 | 84.82 |
| 29 | Dibutyl Sebacate | 2.37 | 15.8 | 7.87 | 56.54 |
| 30 | Citric acid Anhydrous, Granular | 3.16 | 21.07 | 10.51 | 75.50 |
| 31 | Sucralose | 0.03 | 0.2 | 0.10 | 0.75 |
| 32 | Mixed Berry Flavoring (807.1196U) | 0.13 | 0.87 | 0.42 | 3.02 |
| 33 | FD&C Blue #1 Aluminum Lake | 0.06 | 0.4 | 0.18 | 1.32 |
| 34 | Isopropyl Alcohol** | 85 | — | — | — |

| | | | | |
|---|---|---|---|---|
| Total | 100.00 | 100.00 | 100.00 | 718.25 |

| | |
|---|---|
| % solids | 15.00 |

*Opadry Clear YS-1-19025A is composed of 90.9% of Hypromellose 2910, E6 and 9.1% of PEG 400
**removed during processing
**17.3 mg of Ketamine Hydrochloride is equivalent to 15.0 mg of Ketamine

### BIOLOGICAL EXAMPLES

### Example 1. Dual-Route Ketamine and Aspirin in Musculoskeletal Pain Reduction

This example evaluates analgesic efficacy of dual-route ketamine (liquid intraoral ketamine+oral ketamine) taken simultaneously with dual-route aspirin (intraoral aspirin+oral aspirin) for pain management of adult emergency department (ED) patients presenting to the ED with acute musculoskeletal pain.

It is contemplated that the treatment will result in analgesia with a change in pain score at least of 1.3 points on numeric rating pain scale (NRS). The primary outcome of this test is the reduction in participant's pain scores at 60 minutes post medication administration.

Subjects: Patients 18 years of age and older presenting to the ED with acute musculoskeletal painful conditions (traumatic and non-traumatic) with an initial pain score of 5 or more on a standard 11-point (0 to 10) numeric rating scale and requiring oral analgesia as determined by the treating attending physician. Study investigators and research assistants will perform patients' screening and enrollment. All patients will be enrolled at various times of the day when study investigators will be available for patient enrollment and an ED pharmacist will be available for medication preparation

Eligibility Criteria: Patients 18 years of age and older presenting to the ED with acute musculoskeletal painful conditions (traumatic and non-traumatic) with an initial pain score of 5 on a standard 11-point (0 to 10) numeric rating scale. Patients will be awake, alert, oriented to person, place, and time, and will be able to demonstrate understanding of the informed consent process and content. Patients also will have to demonstrate ability to verbalize the nature of any adverse effects they might experience as well as to express their pain severity by using the NRS.

Exclusion Criteria: Patients with: altered mental status, allergy to aspirin and ketamine, pregnant patients, unstable vital signs (systolic blood pressure<90 or >180 mm Hg, pulse rate<50 or >150 beats/min, and respiration rate<10 or >30 breaths/min), inability to provide consent, consumption of Aspirin or NSAID's within 6 hours of arrival to the ED, active PUD, history of GI Hemorrhage, history of renal and hepatic insufficiency, past medical history of alcohol or drug abuse, or schizophrenia.

Design: This is a prospective observational pilot trail evaluating analgesic efficacy and safety of dual-route ketamine in adult patients presenting to the ED of Maimonides Medical Center with acute musculoskeletal painful conditions. Upon meeting the eligibility criteria, patients will be offered to participate in the study.

Data Collection Procedures: Each patient will be approached by a study investigator for acquisition of written informed consent and Health Insurance Portability and Accountability Act authorization after being evaluated by the treating emergency physician and determined to meet study eligibility criteria. When English is not the participant's primary language, a language-appropriate consent form will be used and non-investigator, hospital-employed, trained interpreters or licensed telephone interpreter will assist in acquisition of informed consent. Baseline pain score will be determined with an 11-point numeric rating scale (0 to 10), described to the patient as “no pain” being 0 and “the worst pain imaginable” being 10. A study investigator will record the patient's body weight and baseline vital signs.

The on-duty ED pharmacist will prepare an oral dose of ketamine by using a formulary for parenteral use. The oral dosing regimen of ketamine is 0.5 mg/kg that will be placed in the syringe or a medication cup and sweetener (Ora-sweet®) will be added to offset unpleasant taste of the ketamine. The total dose for aspirin is 325 mg as specified by the sponsor. The research associate will deliver both medications (dual-route aspirin and dual-route ketamine) to the patients' nurse. Study investigators will record pain scores and adverse effects at 15, 30, 60, 90, and 120 minutes. If patients reported a pain numeric rating scale score of 5 or greater and requested additional pain relief, an oral immediate release morphine tablet of 7.5 mg will be given.

All data will be recorded on data collection sheets, including patients' sex, demographics, medical history, and vital signs, and entered into SPSS (version 24.0; IBM Corp) by the research manager. The statistician, who will work independently of any data collection, will conduct statistical analyses.

Patients will be closely monitored for adverse effects during the entire study period (up to 120 minutes) by study investigators. Common adverse effects that are associated with oral ketamine are felling of unreality, dizziness, nausea, vomiting, and sedation. Common adverse effects are associated with dual-route aspirin are nausea, dyspepsia, epigastric discomfort.

Data Analysis: Data analyses will include frequency distributions and independent-sample t-test to assess differences in pain scores at the various intervals. Mixed-model linear regression will be used to compare changes in pain on numeric rating scale across time points.

For categorical outcomes (e.g., complete resolution of pain), X² or Fisher's exact test will be used to compare outcomes at 60 minutes. Based on the validation of a verbally administered rating scale of acute pain in the ED and the comparison of verbal and visual pain scales, this example will use a primary outcome consisting of a minimal clinically meaningful difference of 1.3 between three groups at the 60-minute pain assessment.

Sample Size: Assuming a minimal clinically meaningful difference of 1.3 in change of pain score from the baseline until 60 minutes, given a standard deviation of 3.0, with a one-side 97.5% confidence interval, this example will need 21 subjects for this pilot trial. This example would enroll 25 patients to account for any loss to follow-up.

Expected Outcomes: The primary outcome will include a reduction of pain scores on numeric rating pain scale (NRS) at 60 minutes mark form the baseline. The secondary outcomes will include a need for rescue analgesia and rates of adverse up to 90 minutes. With respect to unique adverse effects of SDK, this example will use Side Effect Rating Scale for Dissociative Anesthetics (SERSDA) and Richmond Agitation Sedation Scale (RASS). SERSDA Scale includes fatigue, dizziness, nausea, headache, feeling of unreality, changes in hearing, mood change, general discomfort, and hallucinations with severity of each graded by patients on a five-point scale, with “0” representing the absence of any adverse effects and “4” representing a severely bothersome side effect. RASS evaluates the severity of agitation and/or sedation in accordance to the nine-point scale with scores ranging from “−4” (deeply sedated) to “0” (alert and calm) to “+4” (combative).

Adverse Events: Dizziness, nausea, vomiting, agitation and or sedation, weakness or fatigue, feelings of unreality, epigastric pain, dyspepsia.

SAE Reporting: Any serious adverse event, requiring intervention, will be reported to the IRB within 24 hours of discovery by the research staff. Less serious adverse events will be reported within a week of discovery. There are known expected outcomes and side effects to the procedures and medications being received and these are the same risks/side effects as the standard of care—these will be reported if they are serious and require intervention.

Timetable: The entire study (from commencement until recruitment of the last patient) will last 12 months. The research team will monitor and record each patient's pain scores and adverse events. The research team, pharmacist, and research manager will be immediately aware and/or notified if a serious adverse event occurs. ED team of physicians and nurses will treat the patient appropriately, and subsequently the adverse effect report will be filed to the IRB.

### Example 2. Comparison of Ketamine/Aspirin With Ketamine Alone

This example compares dual-route ketamine (oral+intraoral) to oral ketamine alone plus oral aspirin alone in adult patients presenting to the ED with acute musculoskeletal pain, in a randomized, double-blind, clinical trial.

Oral formulations of ketamine are not commercially available. The parenteral formulation is given as an oral solution by using an injectable vial. The oral bioavailability of ketamine, defined as area under plasma concentration time curve (AUC), after a single oral dose of 0.5 mg/kg is about one fifth of the availability after an intravenous injection. In a ketamine-naive patient, oral administration of ketamine can start with a single dose of 0.5 mg/kg ketamine racemic mixture or 0.25 mg/kg S-ketamine to evaluate the effect on pain relief and the duration of effect.

Doses can be increased in steps of 0.5 or 0.25 mg/kg according to the efficacy and adverse effects, respectively. The average dosing frequency of 3-4 times daily found in the clinical studies corresponds well with the elimination half-lives of ketamine (2-3 h) and nor-ketamine (4 h). The conversion from parenteral to oral administration in an equipotent dose is complex and is not solely based on a reduced bioavailability. The median conversion rate from subcutaneous to oral ketamine used in the case reports was 1:1.

The primary outcome of this trial is the comparative reduction in participant's pain scores at 60 minutes post-medication administration.

TABLE 4
Example Arms and Interventions

| Arms | Assigned Intervention |
|---|---|
| Ketamine + Aspirin | 0.5 mg/kg of ketamine (oral) + 324 mg of aspirin |
| | (oral) |
| Ketamine alone | 0.5 mg/kg of ketamine (oral + intraoral) |
| (dual-route) | |

Subjects: Patients 18 years of age and older presenting to the ED with acute musculoskeletal painful conditions (traumatic and non-traumatic) with an initial pain score of 5 or more on a standard 11-point (0 to 10) numeric rating scale and requiring oral analgesia as determined by the treating attending physician. Patients' screening and enrollment will be performed by study investigators and research assistants. All patients will be enrolled at various times of the day when study investigators will be available for patient enrollment and an ED pharmacist will be available for medication preparation.

Eligibility Criteria: Patients 18 years of age and older presenting to the ED with acute musculoskeletal painful conditions (traumatic and non-traumatic) with an initial pain score of 5 on a standard 11-point (0 to 10) numeric rating scale. Patients will have to be awake, alert, and oriented to person, place, and time, and will be able to demonstrate understanding of the informed consent process and content. Patients also will have to demonstrate ability to verbalize the nature of any adverse effects they might experience as well as to express their pain severity by using the NRS.

Exclusion Criteria: Patients with altered mental status, allergy to aspirin and ketamine, pregnant patients, unstable vital signs (systolic blood pressure<90 or >180 mm Hg, pulse rate<50 or >150 beats/min, and respiration rate<10 or >30 breaths/min), inability to provide consent, consumption of Aspirin or NSAID's within 6 hours of arrival to the ED, active PUD, history of GI Hemorrhage, history of renal and hepatic insufficiency, past medical history of alcohol or drug abuse, or schizophrenia.

Design: This is a prospective, randomized, double-blind trial comparing analgesic efficacy and safety of the treatments in patients presenting to the ED of Maimonides Medical Center with acute musculoskeletal pain. Upon meeting the eligibility criteria, patients will be randomized into one of the two study arms: 325 mg dual-route aspirin+1 mg/kg oral ketamine, and oral ketamine alone at 1 mg/kg.

Data Collection Procedures: Each patient will be approached by a study investigator for acquisition of written informed consent and Health Insurance Portability and Accountability Act authorization after being evaluated by the treating emergency physician and determined to meet study eligibility criteria. When English is not the participant's primary language, a language-appropriate consent form will be used and non-investigator, hospital-employed, trained interpreters or licensed telephone interpreter will assist in acquisition of informed consent. Baseline pain score will be determined with an 11-point numeric rating scale (0 to 10), described to the patient as “no pain” being 0 and “the worst pain imaginable” being 10. A study investigator will record the patient's body weight and baseline vital signs. All data will be recorded on data collection sheets, including patients' sex, demographics, medical history, and vital signs, and entered into SPSS (version 24.0; IBM Corp) by the research manager. Confirmation of written consent acquisition for all participants, and statistical analyses will be conducted by the statistician (Michael Silver), who will work independently of any data collection.

The on-duty ED pharmacist will prepare an oral dose of ketamine by using an injectable form for parenteral use. The on-duty ED pharmacist will prepare a syringe/medication cup containing: the combo group (Combo): 325 mg and 0.5 or 1 mg/kg oral dose ketamine; oral ketamine (OK) group: matching placebo tablets and 0.5 or 1 mg/kg oral dose ketamine according to the predetermined randomization list, which will be created in SPSS (version 24; IBM Corp, Armonk, NY) with block randomization of every 10 participants. The research associate will deliver syringes/medication cups to the patients' nurse who will administer medication. Study investigators will record pain scores and adverse effects at 30, 60, 90, and 120 minutes. If patients reported a pain numeric rating scale score of 5 or greater and requested additional pain relief, an oral immediate release morphine tablet of 7.5 mg will be given. Patients will be closely monitored for adverse effects during the entire study period (up to 120 minutes) by study investigators. Common adverse effects that are associated with OK are felling of unreality, dizziness, nausea, vomiting, and sedation. Common adverse effects are associated with Combo are nausea, dyspepsia, epigastric discomfort, heartburn, dizziness.

Data Analysis: Data analyses will include frequency distributions and independent-sample t-test to assess differences in pain scores at the various intervals. Mixed-model linear regression will be used to compare changes in pain numeric rating scale across time points.

For categorical outcomes (e.g., complete resolution of pain), a X² or Fisher's exact test will be used to compare outcomes at 60 minutes. Based on the validation of a verbally administered rating scale of acute pain in the ED and the comparison of verbal and visual pain scales, we will use a primary outcome consisting of a minimal clinically meaningful difference of 2 between two groups at the 60-minute pain assessment.

Contemplated Outcomes: The primary outcome will include a reduction of pain scores on numeric rating pain scale (NRS) at 60 minutes mark form the baseline. The secondary outcomes will include a need for rescue analgesia and rates of adverse up to 120 minutes. With respect to unique adverse effects of SDK, we will use Side Effect Rating Scale for Dissociative Anesthetics (SERSDA) and Richmond Agitation Sedation Scale (RASS) (ref) SERSDA Scale includes fatigue, dizziness, nausea, headache, feeling of unreality, changes in hearing, mood change, general discomfort, and hallucinations with severity of each graded by patients on a five-point scale, with “0” representing the absence of any adverse effects and “4” representing a severely bothersome side effect. RASS evaluates the severity of agitation and/or sedation in accordance to the nine-point scale with scores ranging from “−4” (deeply sedated) to “0” (alert and calm) to “+4” (combative).

SAE Reporting: Any serious adverse event, requiring intervention, will be reported to the IRB within 24 hours of discovery by the research staff. Less serious adverse events will be reported within a week of discovery. There are known expected outcomes and side effects to the procedures and medications being received and these are the same risks/side effects as the standard of care—these will be reported if they are serious and require intervention.

Timetable: The entire study (from commencement until recruitment of the last patient) will last 12 months. The research team will monitor and record each patient's pain scores and adverse events. The research team, pharmacist, and research manager will be immediately aware and/or notified if a serious adverse event occurs. The patient will be treated appropriately by ED team of physicians and nurses, and subsequently the adverse effect report will be filed to the IRB.

### Example 3. Management of Headache

This example compares the combination of dual-route aspirin and dual-route ketamine to Rimegepant (Nurtec®, Biohaven Pharmaceuticals, New Haven, CT) for managing acute headache in the ED in a randomized, open-label, clinical trial.

Nurtec (Rimegepant) is an orally administered small molecule CGRP receptor antagonist with efficacy in the acute treatment of migraine. Following oral administration of NURTEC Orally Disintegrating Tablet, rimegepant is absorbed with the maximum concentration at 1.5 hours. The absolute oral bioavailability of rimegepant is approximately 64%. Rimegepant is primarily metabolized by CYP3A4 and to a lesser extent by CYP2C9. Rimegepant is primarily eliminated in unchanged form (˜77% of the dose) with no major metabolites (i.e., >10%) detected in plasma. The most common adverse reaction are nausea (2% in patients who received NURTEC ODT compared to 0.4% of patients who received placebo). Hypersensitivity, including dyspnea and severe rash, occurred in less than 1% of patients treated with NURTEC ODT.

NURTEC ODT 75 mg orally disintegrating tablets are white to off-white, circular, debossed with the symbol, and supplied in cartons containing a blister pack of 8 orally disintegrating tablets. Each ODT contains 75 mg rimegepant.

This example contemplates that the administration of a combination of dual-route aspirin and oral ketamine would provide similar analgesic efficacy to Nurtec with respect to analgesic efficacy at 60 min and 120 minutes in ED patients with acute headache.

Subjects: Patients 18 years of age and older presenting to the ED with acute headache (defined as HA lasting no more than 1 week) and an initial pain score of 5 or more on a standard 11-point (0 to 10) numeric rating scale and requiring oral analgesia as determined by the treating attending physician. Patients' screening and enrollment will be performed by study investigators and research assistants. All patients will be enrolled at various times of the day when study investigators will be available for patient enrollment and an ED pharmacist will be available for medication preparation.

Eligibility Criteria: Patients 18 years of age and older presenting to the ED with acute headache (<7 days) and an initial pain score of 5 on a standard 11-point (0 to 10) numeric rating scale. Patients will have to be awake, alert, and oriented to person, place, and time, and will be able to demonstrate understanding of the informed consent process and content. Patients also will have to demonstrate ability to verbalize the nature of any adverse effects they might experience as well as to express their pain severity by using the NRS.

Exclusion Criteria: Patients with altered mental status, allergy to aspirin/ketamine/rimegepant, pregnant patients, unstable vital signs (systolic blood pressure<90 or >180 mm Hg, pulse rate<50 or >150 beats/min, and respiration rate<10 or >30 breaths/min), inability to provide consent, consumption of Aspirin/NSAID's within 6 hours of arrival to the ED, or acetaminophen within 4 hours of arrival to the ED, active PUD, history of GI Hemorrhage, history of renal and hepatic insufficiency, past medical history of alcohol or drug abuse, or schizophrenia, as well as clinical findings concerning for acute intracranial process, acute infections process, or vascular catastrophe, pregnant patients and breastfeeding patients.

Design: This is a prospective, open-label, equivalence trial evaluating and comparing analgesic efficacy and safety of a combination of dual-route aspirin and oral ketamine, and Nurtec in adult patients presenting to the ED of Maimonides Medical Center with acute headache. Upon meeting the eligibility criteria, patients will be offered to participate in the study.

Data Collection Procedures: Each patient will be approached by a study investigator for acquisition of written informed consent and Health Insurance Portability and Accountability Act authorization after being evaluated by the treating emergency physician and determined to meet study eligibility criteria. When English will not be the participant's primary language, a language-appropriate consent form will be used and non-investigator, hospital-employed, trained interpreters or licensed telephone interpreter will assist in acquisition of informed consent. Baseline pain score will be determined with an 11-point numeric rating scale (0 to 10), described to the patient as “no pain” being 0 and “the worst pain imaginable” being 10. A study investigator will record the patient's body weight and baseline vital signs. All data will be recorded on data collection sheets, including patients' sex, demographics, medical history, and vital signs, and entered into SPSS (version 24.0; IBM Corp) by the research manager. Confirmation of written consent acquisition for all participants, and statistical analyses will be conducted by the statistician (Michael Silver), who will work independently of any data collection.

Study Set-up. The on-duty ED pharmacist will prepare medications in the following fashion: the combination of dual-route aspirin (325 mg) and oral ketamine (0.85 or 1 mg/kg) (Combo) and Nurtec that are provided by the sponsor will be placed in the medication cups according to a randomization list generated by the research manager by SPSS (version 24.0; IBM Corp, Armonk, NY). The oral dose of ketamine will be prepared by using an injectable form of ketamine at 0.85 or 1 mg/kg dose and matching (by volume) placebo (normal saline). This weight-based dose of ketamine and saline placebo will be placed in the syringe and sweetener will be added to offset the bitter taste of ketamine. The Combo group will receive the combination medication cup and oral ketamine syringe, and Nurtec group will receive Nurtec ODT blister containing 75 mg of rimegepant in the medication cup and oral placebo syringe.

The research associate will deliver both syringes and medication cups to the patients' nurse who will administer medication. Study investigators will record pain scores and adverse effects at 30, 60, 90, and 120 minutes. If patients reported a pain numeric rating scale score of 5 or greater and requested additional pain relief, the rescue analgesic(s) and routes will be chosen upon treating attending's discretion. Patients will be closely monitored for adverse effects during the entire study period (up to 120 minutes) by study investigators. Common adverse effects that are associated with oral ketamine are felling of unreality, dizziness, nausea, vomiting, and sedation. Common adverse effects that are associated with Combo are nausea, dyspepsia, epigastric discomfort. Common adverse effect that is associated with Nurtec include nausea.

Data Analysis: Data analyses will include frequency distributions and independent-sample t-test to assess differences in pain scores at the various intervals. Mixed-model linear regression will be used to compare changes in pain numeric rating scale across time points. For categorical outcomes (e.g., complete resolution of pain), a X² or Fisher's exact test will be used to compare outcomes at 60 and 120 minutes. Based on the validation of a verbally administered rating scale of acute pain in the ED and the comparison of verbal and visual pain scales, we will use a primary outcome consisting of a minimal clinically meaningful difference of 2 points between two groups at the 60-minute and 120-minute pain assessment.

Contemplated Outcomes: The primary outcome will include a comparative reduction of pain scores on numeric rating pain scale (NRS) at 60 minutes from the baseline. The secondary outcomes will include a need for rescue analgesia, rates of adverse effects, and change in pain score up to 120 minutes.

SAE Reporting: Any serious adverse event, requiring intervention, will be reported to the IRB within 24 hours of discovery by the research staff. Less serious adverse events will be reported within a week of discovery. There are known expected outcomes and side effects to the procedures and medications being received and these are the same risks/side effects as the standard of care—these will be reported if they are serious and require intervention.

### Example 4. Oral VTS-K (Combination of VTS-Aspirin and Oral Ketamine) as an Adjunct to Oral Antidepressant Therapy in Treatment of Major Depressive Disorder

This example tests the use of ketamine in treating depression. It is contemplated that 486 mg of aspirin (dual-route intraoral/oral administration) and 1.2 mg/kg ketamine (dual-route intraoral/oral administration) as an adjunct to oral antidepressant treatment will result in reduction of depressive symptoms based on the change in score from baseline to day 7 after initial dose on the Montgomery-Åsberg Depression Rating Scale (MADRS).

Subjects: This is a prospective observational proof of concept study of medically stable (based on the physical examination, history, and vital signs) adults patients 18 years of age and older with a diagnosis of major depressive disorder without psychotic features according to DSM-IV-TR criteria presenting to the psychiatric clinic for evaluation. Patients to have a score≥22 on the Montgomery-Åsberg Depression Rating Scale (MADRS) on day 1 before dosing. Participants ought to agree to continue oral antidepressant therapy they were receiving prior to the enrollment into the study.

### Eligibility Criteria:

Adult patients with TRD with MADRS score>22 upon presentation to the clinic. Participants ought to agree to standard-of-care treatment with one or more non-investigational antidepressants.

Participants ought to agree to continue oral antidepressant therapy they were receiving prior to the enrollment into the study. All participants must agree to provide and informed consent.

### Exclusion Criteria:

Adult patients with recent or current suicidal ideation with an intent to act, homicidal ideations with an intent to act, intellectual disability, major depressive disorder with psychosis, posttraumatic stress disorder, obsessive-compulsive disorder, substance use disorder, antisocial personality disorder, borderline personality disorder, or a current or past diagnosis of a psychotic disorder altered mental status, allergy to aspirin and ketamine, pregnant patients, unstable vital signs (systolic blood pressure<90 or >180 mm Hg, pulse rate<50 or >150 beats/min, and respiration rate<10 or >30 breaths/min), consumption of Aspirin or NSAID's within 6 hours of arrival to the ED, active PUD, history of GI Hemorrhage, and history of renal and hepatic insufficiency.

### Design:

This is a prospective observational proof of concept study of adult patients 18 years of age and older with a diagnosis of TRD without psychotic features according to DSM-IV-TR criteria presenting to the psychiatric clinic for evaluation. Eligible participants will receive 486 mg of aspirin (dual-route intraoral/oral administration) and 1.2 mg/kg ketamine (dual-route intraoral/oral administration) as an adjunct to oral antidepressant treatment twice a week for 1 week (on day 1 and 4 for the week). All participants will be observed for 4 hours in the outpatient setting after receiving the medication and their depressive symptoms will be assessed at 4-hour mark by MADRS Scale and QIDS-SR scale (Quick Inventory of Depressive Symptomatology-Self Report) and their adverse effect related to VTS=K administration will be assessed by using the Clinician Administered Dissociative State Scale (CADSS) and MOAA/S scale and RASS scale.

### Study Setup

The on-duty pharmacist will prepare an oral dose of ketamine by using an injectable form for parenteral use. The on-duty ED pharmacist will prepare a syringe/medication cup containing the medication according to the predetermined list, which will be created in SPSS (version 24; IBM Corp, Armonk, NY). The oral sweetener solution of 1ml will be added to the syringe containing ketamine. The research pharmacist will deliver syringes/medication cup to the patients' nurse who will administer medication. Subjects will be instructed to first suck off the outer layer of aspirin for about 30-60 seconds and then swallow the tablet. The oral antidepressant medication will be initiated or optimized for all participants on day 1. Participants taking a recently initiated antidepressant at screening could continue the antidepressant at the same dosage during treatment with study drug. Study investigators will record VS, and adverse effects at 30 minute, 60 minutes, 2 hours, and 4 hours. Patients will be closely monitored for adverse effects during the entire observation period (for up to 2-4 hours) by study investigators.

### Efficacy Assessments

Research associates and study investigators will assess depressive symptom severity with the Montgomery-Åsberg Depression Rating Scale (MADRS) on day one and on day 4 and day 7 (for days 1 and 4 this will include pre-dose, 40 minutes, 120 minutes, and 240 minutes post-dose). Patients will also be screened using the Beck Scale for Suicide Ideation and their sense of hopelessness using the Beck Hopelessness Scale before dosing and 4 hours after dosing on day one and pre-dosing on days 4, and 7.

### Safety Assessments

Vital signs will be checked before medication administration and 1, 2, 4 hours post-drug administration during the study period. Adverse events will be monitored throughout the study. The Clinician-Administered Dissociative States Scale (CADSS) will be administered before dosing and at 40 minutes, 2 hours, and 4 hours after dosing on day 1 and day 4. In addition, a Modified Observer's Alertness/Sedation Scale (MOAA/S) will be used to assess the level of sedation.

Common adverse effects that are associated with oral ketamine are felling of unreality, dizziness, nausea, vomiting, sedation, and dissociation. Common adverse effects are associated with VTS-Aspirin are nausea, dyspepsia, epigastric discomfort,

Data Collection Procedures: Each patient will be approached by a study investigator for acquisition of written informed consent and Health Insurance Portability and Accountability Act authorization after being evaluated by the treating psychiatry physician and determined to meet study eligibility criteria. When English will not be the participant's primary language, a language-appropriate consent form will be used and non-investigator, hospital-employed, trained interpreters or licensed telephone interpreter will assist in acquisition of informed consent. Baseline Depression score via MADRS will be recorded by a study investigator. All data will be recorded on data collection sheets, including patients' sex, demographics, medical history, and vital signs, and entered into SPSS (version 24.0; IBM Corp) by the research manager. Confirmation of written consent acquisition for all participants, and statistical analyses will be conducted by the statistician who will work independently of any data collection.

Data Analysis: Data analyses will include mixed-effect model with repeated measures with baseline MADRS score as covariate; time by treatment interaction as fixed effect and patient as random effect.

### Expected Outcomes:

The primary outcome will include a change in depressive symptoms on the Montgomery-Åsberg Depression Rating Scale (MADRS) from the baseline (pre-dose day 1) and day 7.

The secondary outcomes will include: a change in depressive symptoms on the Montgomery-Åsberg Depression Rating Scale (MADRS) from the baseline (pre-dose on day 1 and 4) and 4 hours post-medication administration.

Rates of Side effects will be reported by using the Clinician-Administered Dissociative States Scale (CADSS), Side Effect Rating Scale for Dissociative Anesthetics (SERSDA), and Modified Observer's Alertness/Sedation Scale (MOAA/S). The SERSDA scale includes fatigue, dizziness, nausea, headache, feeling of unreality, changes in hearing, mood change, general discomfort, and hallucinations with severity of each graded by patients on a five-point scale, with “0” representing the absence of any adverse effects and “4” representing a severely bothersome side effect.

SAE Reporting: Any serious adverse event, requiring intervention, will be reported to the IRB within 24 hours of discovery by the research staff. Less serious adverse events will be reported within a week of discovery. There are known expected outcomes and side effects to the procedures and medications being received and these are the same risks/side effects as the standard of care—these will be reported if they are serious and require intervention.

### Example 5. Therapeutic Effects of Dual Ketamine/Aspirin

This example presents the results from a trial as outlined in Example 1, in comparison with data collected from similar trials with other agents and the same testing protocol and pain measurement scales, in the same medical center by the same research team and same statistical methods. The testing agents and dosages are listed in Table 5.

TABLE 5
Agents and Doses

| No. | Agent | Dose |
|---|---|---|
| 1 | Morphine (IV) | 0.1 mg/kg |
| 2 | Morphine Sulfate Immediate Release (MSIR) (oral) | 15 mg |
| 3 | Percocet (oral) | 10 mg |
| 4 | Dual route ketamine + aspirin (intraoral + oral) | 0.5 mg/kg |
| | | ketamine |
| 5 | Ketamine (IV) | 0.3 mg/kg |
| 6 | Nebulized ketamine | Mixed dose |
| 7 | Ibuprofen (oral) | Mixed dose |
| 8 | Ketorolac (IV) | |

The efficacy to reduce acute musculoskeletal pain by each agent was measured as described in Example 1. The results are presented in FIG. 1. As expected, IV morphine had the highest efficacy, resulting in a 5.1-point reduction of acute musculoskeletal pain. The least efficacious was oral ibuprofen (2-point reduction). IV ketamine and intranasal (nebulized) ketamine were both effective, achieving a reduction of about 4 points, similar to oral morphine and Percocet.

Quite unexpectedly, the dual-route ketamine and dual-route aspirin achieved a 3.84-point reduction of acute musculoskeletal pain. As explained above, it has been demonstrated that the oral bioavailability of ketamine is about ⅕ of the availability via IV injection. Here, the measured efficacy of dual-route ketamine (0.5 mg/kg) and aspirin (3.84 points) is equivalent to that of IV ketamine (0.3 mg/kg) (3.8 points). Therefore, these data suggest that the dual-route ketamine (with aspirin) at 0.5 mg/kg is equivalent to conventional oral ketamine at 1.5 mg/kg. This is a three-fold increase of efficacy as compared to the conventional oral ketamine.

A typical side effect of ketamine is sedation, which was measured with a Richmond Agitation-Sedation Scale (RASS) as described in Table 6A. The results are compared to published ones that either used the RASS scale or a MOAA/S scale (Modified Observer's Assessment of Alertness/Sedation) which is described in Table 6B. The published data include those from Motov 2017 (Motov S. et al., Ann Emerg Med. 2017 August; 70(2):177-184), Motov 2019 (Motov S. et al., Am J Emerg Med, 2019 February; 37(2):220-227), and the drug label of Spravato® esketamine (intranasal ketamine).

TABLE 6A
Sedation Measurement Scores - RASS

| Score | Description |
|---|---|
| +4 | Combative |
| +3 | Very agitated |
| +2 | Agitated |
| +1 | Restless |
| 0 | Alert and calm |
| −1 | Drowsy |
| −2 | Light sedation |
| −3 | Moderate sedation |
| −4 | Deep sedation |
| −5 | Unarousable sedation |

TABLE 6B
Sedation Measurement Scores - MOAA/S

| Score | Description |
|---|---|
| 5 | Responds readily to name spoken in normal tone |
| 4 | Lethargic response to name spoken in normal tone |
| 3 | Responds only after name is called loudly and/or repeatedly |
| 2 | Responds only after mild prodding or shaking |
| 1 | Responds only after painful trapezius squeeze |
| 0 | No response after painful trapezius squeeze |

The results are shown in FIG. 2, along with control data from the publications. Only 8% patients who received the dual-route ketamine and aspirin had any sedation side effect, and only 4% reported moderate or above sedation side effects (RASS<=−3 or MOAA/S<=3). Such rates are considerably lower than both IV ketamine and intranasal ketamine. Given that the dual-route ketamine and aspirin was as efficacious as IV and intranasal ketamine, its greatly reduced sedation side effect was truly a surprise.

Another common side effect of ketamine is dissociation, which was measured with a Side Effects Rating Scale of Dissociative Anesthetics (SERSDA) scale as described in Table 7A. The results are compared to published ones that either used the SERSDA scale or a Clinician-Administrated Dissociative States Scale (CADSS) which is described in Table 7B. The published data include those from Motov 2017, Motov 2019, and the drug label of Spravato® esketamine (intranasal ketamine).

TABLE 7A
Dissociation Measurement Scale - SERSDA

| Score | Description |
|---|---|
| 0 | No change |
| 1 | Weak |
| 2 | Modest |
| 3 | Bothersome |
| 4 | Very Bothersome |

TABLE 7B
Dissociation Measurement Scale - CADSS

27 questions
0-4 points each
>4 increase “positive”

The results are shown in FIG. 3, along with control data from the publications. Only 4% patients who received the dual-route ketamine and aspirin had any dissociation side effect (SERSDA Unreality; *CADSS>4 pt incurrence), and none (0%) reported significant dissociation side effects (SERSDA Unreality: Bothersome+; *CADSS>10 pt incurrence). Such rates are considerably lower than both IV ketamine and intranasal ketamine. Given that the dual-route ketamine and aspirin was as efficacious as IV and intranasal ketamine, its greatly reduced dissociation side effect was again truly a surprise.

### Example 6. Additional Testing of Different Combinations

This example provides the results of additional clinical testing of various compositions of the instant disclosure, in terms of their pain reduction efficacy and associated adverse effects.

This new study was an extension of the study in Example 5, at the same site, also with patients with acute musculoskeletal pain in the ER (e.g., acute back pain, or a wrist fracture). The tested formulations are described below.

In a first analysis, data collected from only patients having fractures were analyzed. They were divided into an aspirin/ketamine (Any ASA/KET) group (N=11) and a dual-route (intraoral and oral) ketamine (Dual Route KET) group (N=4). Primarily, the safety endpoint was examined defined as any SERSDA scores of 3 or higher (i.e., moderately or severely “bothersome”). As shown in FIG. 4, none of the patients that received both aspirin and ketamine (Any ASA/KET) had any moderate or severe adverse events, while 25% of the patients receiving dual-route ketamine (Dual Route KET) had adverse events.

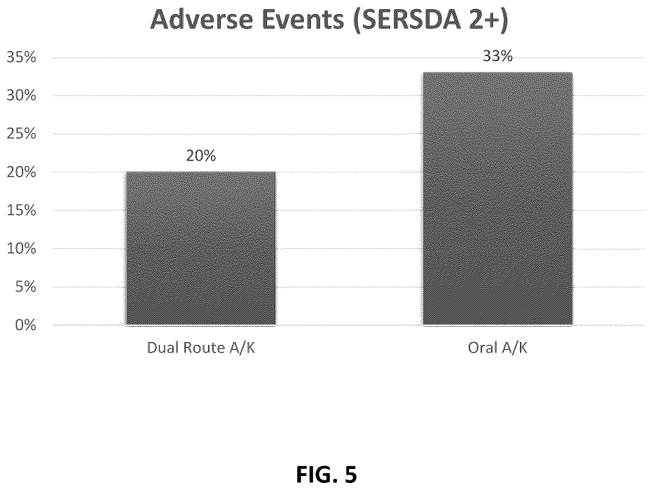

The aspirin/ketamine group was further divided, into a dual route aspirin+dual route ketamine (Dual Route A/K) arm and an oral aspirin+oral ketamine (Oral A/K) arm. In this comparison, all adverse events of SERSDA2+ were examined. As shown in FIG. 5, the Oral A/T arm had significantly higher (65% more) SERSDA2+ events than the Dual Route A/K arm.

In the second analysis, also only with patients having fractures, patients with any combination of ASA/KET (Any ASA/KET) were compared to patients that received dual-route (intraoral and oral) ketamine (Dual Route KET). As shown in FIG. 6, the pain reduction efficacy of the ASA/KET combination was about 36% higher than the Dual Route KET arm. When the ASA/KET group was divided into two subgroups (i.e., Dual Route A/K and oral aspirin+oral ketamine (Oral A/K)), both subgroups outperformed dual-route ketamine (FIG. 7).

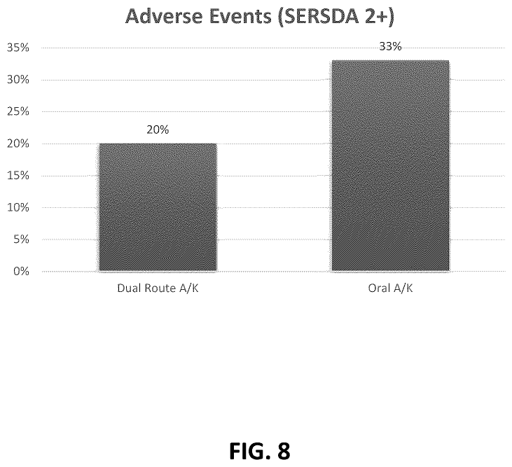

In the third analysis, patients that received oral aspirin and oral ketamine (Oral A/K, N=25) were compared to those who received Dual Route A/K (N=10). None of the patients experienced SERSDA3+. The comparison of SERSDA2+ events is shown in FIG. 8, which shows that 65% more ASA/KET patients had adverse events than Dual Route A/K patients.

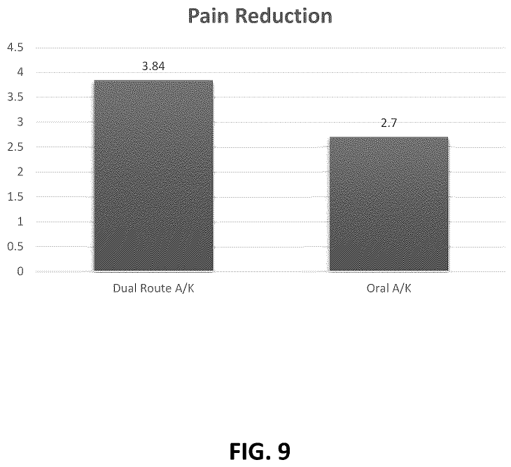

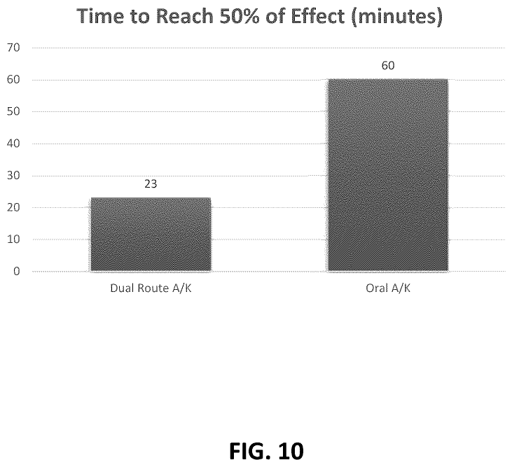

In terms of pain reduction, Dual Route A/K patients had 42% more pain reduction, 3.84 vs. 2.7, as shown in FIG. 9. Also, in terms of time to onset (50% of pain reduction), the Dual Route A/K arm experienced much quicker pain relief, 23 minutes vs. 60 minutes, as shown FIG. 10.

### Example 7. Potency and Long Acting Effects of the Formulations

In this example, various regimens of ketamine and aspirin were tested again in a trial in an urban medical center's emergency department.

The tested regimens included oral ketamine (0.5 mg/kg)+oral aspirin (324 mg) (“K1/A1”), oral ketamine+intraoral ketamine (0.5 mg/kg combined, “K2”), and dual-route ketamine (0.5 mg/kg combined)+dual-route aspirin (324 mg combined) (“K2/A2”). Patients presenting with acute pain with a pain score of at least 5 on a 0-10 point scale were included in the study (8 patients). Pain measurements, as well as sedation and dissociation scale measurements, were taken at 0, 15, 30, 60, 90 and 120 minutes.

The data was compared with best existing versions of ketamine that are in the public domain, including Wafermine by iX Biopharma, an oral sublingual ketamine used for pain management, at 70 mg and 100 mg dosages, and IV ketamine. Clinical data for Wafermine are available through Phase 2b FDA studies (80-120 patients). In order to compare patient populations best, given that Wafermine studies were in post-operation molar extraction, this example specifically analyzed patients who presented with acute pain due to fracture (excluding other potentially less comparative types of pain, such as acute on chronic back pain).

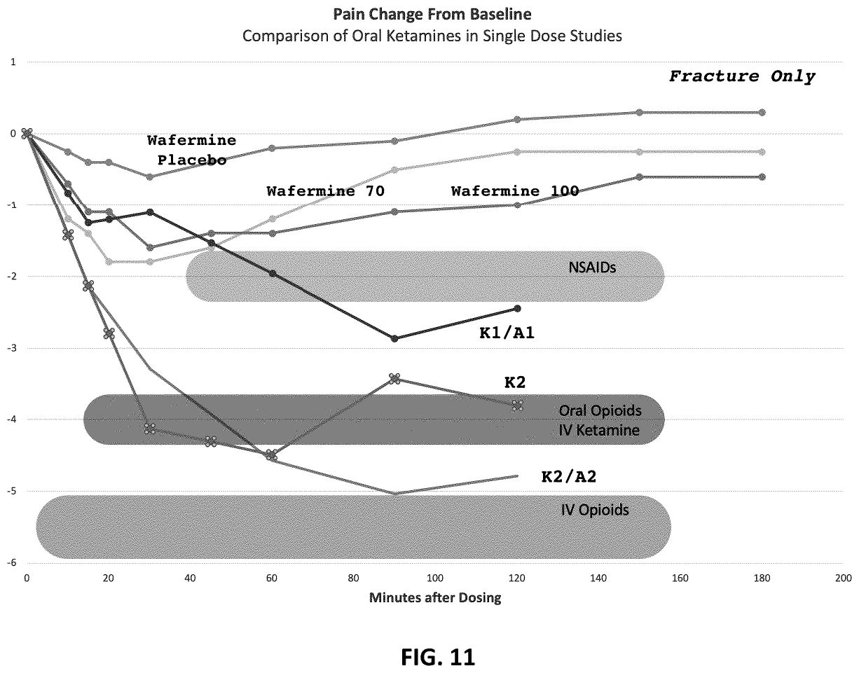

Wafermine is currently considered the most advanced “oral ketamine for pain”. Its efficacies (FIG. 11), however, are generally low (lower than 2 points reduction) and short-acting. This is likely attributed to the low amounts of ketamine used due to concerns of side effects for potential outpatient use.

As shown in FIG. 11, the K2 regimen (oral ketamine+intraoral ketamine) exhibited surprising and clinically significant improvement over Wafermine. Its pain reduction peaked over 4 points (at about 60 minutes) and stayed highly efficacious through the end of study (120 mins).

Interestingly, the K1/A1 regimen also exhibited stronger and longer pain reduction as compared to Wafermine (surprising and clinically meaningful). Its pain reduction peaked at about 90 minutes with a close to 3-point reduction (FIG. 11).

The K2/A2 regimen apparently outperformed all other ketamine regimens including IV ketamine and was only less potent than IV opioids (FIG. 11). K2/A2 has a slightly stronger pain reduction than K2 (5 vs 4.4), but also longer acting than K2, similar to K1/A1 (peak pain reduction at 90 mins, and still very strong pain reduction when the study ended at 120 mins). IV ketamine can be a stronger pain reducer. Its pain reduction effect, however, peaks at about 5 mins. Moreover, IV ketamine comes with very high side effects.

This example, therefore, further confirms the efficacies of the ketamine regimens of the instant disclosure.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A pharmaceutical composition comprising ketamine, wherein, upon oral administration to a subject, a first portion disintegrates or dissolves intraorally providing rapid release of ketamine, and a second portion is substantially more difficult than the first portion to disintegrate or dissolve intraorally but is ingestible and releases ketamine in the gastrointestinal tract of the subject.

2. The pharmaceutical composition of claim 1, comprising:
(i) a core composition comprising ketamine; and
(ii) a coat comprising ketamine and enclosing the core composition.
wherein the coat (ii) forms the first portion which disintegrates or dissolves intraorally providing rapid release of ketamine, and the core composition (i) forms the second portion that is substantially more difficult than the first portion to disintegrate or dissolve intraorally but is ingestible and releases ketamine in the gastrointestinal tract.

3. The pharmaceutical composition of claim 2, wherein the core composition comprises ketamine, microcrystalline cellulose, pregelatinized starch, citric acid anhydrous, croscarmellose sodium, colloidal silicon dioxide, and stearic acid.

4. The pharmaceutical composition of claim 2, wherein the core composition comprises about 3% w/w to about 10% w/w of ketamine, wherein % w/w refers to percent by weight of the total weight of the core composition.

5. The pharmaceutical composition of claim 2, wherein the core composition is a compressed tablet.

6. The pharmaceutical composition of claim 2, wherein the core composition is a capsule.

7. The pharmaceutical composition of claim 2, wherein the coat comprising ketamine is substantially free of an alcohol.

8. The pharmaceutical composition of claim 2, wherein the coat comprising ketamine further comprises a binder, a plasticizer and a disintegrant.

9. The pharmaceutical composition of claim 8, wherein the binder comprises hydroxypropyl methylcellulose (HPMC), ethyl cellulose, methyl cellulose, hydroxypropyl cellulose, or any combination thereof.

10. The pharmaceutical composition of claim 8, wherein the binder comprises hydroxypropyl methylcellulose.

11. The pharmaceutical composition of claim 8, wherein the binder comprises methyl cellulose, and hydroxypropyl cellulose.

12. The pharmaceutical composition of claim 8, wherein the plasticizer is dibutyl sebacate (DBS), dimethyl sebacate (DMS), dioctyl sebacate (DOS), triethyl citrate, triacetin, acetyl tributyl citrate, tributyl citrate, polyethylene glycol, or any combination thereof.

13. The pharmaceutical composition of claim 8, wherein the disintegrant is microcrystalline cellulose (MCC).

14. The pharmaceutical composition of claim 2, wherein the coat comprising ketamine is prepared by spray coating a substrate with an aqueous solution comprising ketamine, a binder, and a disintegrant.

15. The pharmaceutical composition of claim 2, wherein the composition optionally further comprises sweeteners, pigments, and flavoring agents in the core composition, and/or the coat comprising ketamine.

16. The pharmaceutical composition of claim 2, wherein up to 50% of the total ketamine in the composition is released intraorally.

17. A dual release pharmaceutical composition comprising ketamine, wherein, upon oral administration to a subject, a first portion disintegrates or dissolves intraorally providing rapid release of ketamine, and a second portion is substantially more difficult than the first portion to disintegrate or dissolve intraorally but is ingestible and releases ketamine in the gastrointestinal tract of the subject,
wherein the composition comprises:
(i) a core composition comprising:
(i-a) about 3% w/w to about 10% w/w ketamine;
(i-b) about 60% w/w to about 80% w/w microcrystalline cellulose;
(i-c) about 10% w/w to about 20% w/w of pregelatinized starch;
(i-d) about 1% w/w/to about 4% w/w croscarmellose sodium;
(i-f) about 0.5% w/w to about 1.5% w/w silicon dioxide; and
(i-g) about 0.1% w/w to about 0.5% w/w stearic acid;
wherein the % w/w is based on the total weight of the core composition;
(ii) a coat comprising ketamine and enclosing the core composition, and providing about 10% solid weight gain to the core composition (i), wherein the coat comprises
(ii-a) about 1% w/w about 5% w/w ketamine;
(ii-b) about 1% w/w about 5% w/w hydroxypropyl methylcellulose;
(ii-c) about 0.1% w/w to about 0.5% w/w polyethylene glycol;
(ii-d) about 1% w/w to about 5% w/w microcrystalline cellulose;
(ii-e) about 0.01% w/w to about 0.05% w/w sweetener;
(ii-f) about 0.01% w/w to about 0.1% w/w flavoring agent; and
(ii-g) about 0.01% w/w to about 0.05% w/w pigment;
wherein the % w/w is based on the weight of the coat. wherein the coat (ii) forms the first portion which disintegrates or dissolves intraorally providing rapid release of ketamine, and the core composition (i) forms the second portion that is substantially more difficult than the first portion to disintegrate or dissolve intraorally but is ingestible and releases ketamine in the gastrointestinal tract.

18. A method for treating acute pain, headache, depression, or cannabinoid hyperemesis syndrome exacerbations in a patient, comprising orally administering an effective amount of a pharmaceutical composition of claim 2 to the patient.
